(19) United States
(12) Patent Application Publication
(10) Pub. No.: US 2025/0367102 A1
(43) Pub. Date: Dec. 4, 2025

(54) PERSONAL CARE COMPOSITION COMPRISING A BIODEGRADABLE CATIONIC POLYMER

(71) Applicant: THE PROCTER & GAMBLE COMPANY, Cincinnati, OH (US)

(72) Inventors: Eric Scott JOHNSON, Hamilton, OH (US); Brennan Alexander SCHILLING, Cincinnati, OH (US); Brandon Scott LANE, Hamilton, OH (US); Parth Rajesh SHAH, Mason, OH (US)

(21) Appl. No.: 19/228,212

(22) Filed: Jun. 4, 2025

Related U.S. Application Data
(60) Provisional application No. 63/655,877, filed on Jun. 4, 2024.

(51) Int. Cl. A61K 8/73; A61K 8/49; A61Q 5/12

(52) U.S. Cl. CPC A61K 8/73; A61K 8/4926; A61Q 5/12

(57) ABSTRACT

The present invention is directed to a personal care composition comprising from about 8 to about 20% of one or more surfactants; from about 0.01 to about 10% of scalp care active; from about 0.01 to about 5% of one or more cationic polymer; from about 0.1% to about 5% of a cationically modified poly alpha-1,6-glucan ether compound; and wherein the composition has an increase in deposition of the scalp care active when compared to a control composition with no cationically modified poly alpha-1,6-glucan ether compound.

### FIELD OF THE INVENTION

The invention relates to personal care compositions comprising a biodegradable, low-molecular weight, cationic polymer. More specifically, the invention relates to a personal care composition comprising a low molecular weight cationic modified poly alpha-1,6-glucan ether compound, which provides an increase in scalp care active deposition.

### BACKGROUND OF THE INVENTION

Cationic polymers are often used in cleansing formulations to provide wet conditioning as well as improve the deposition efficiency of actives, such as scalp care actives. Cationic polymers must be compatible with other ingredients in the composition, exhibit some degree of salt tolerance, and have minimal effect on stability. Historically there has been a performance trade-off as cationic polymers are made more biodegradable (i.e., becoming less efficient) yielding an opportunity for invention. Furthermore, conventional non-biodegradable cationic polymers exhibit a maximum allottable use level until unwanted feel characteristics emerge (i.e., hair feeling coated, sticky, heavy). With low molecular weight biodegradable polymers, this maximum level is much greater, opening the possibilities of more benefit spaces.

The present invention is related to cationically modified poly alpha-1,6-glucan ether compounds for cationic polymers that have passing biodegradation data for application in cleansing. Cationic Polymers may provide hair conditioning and active delivery benefits, where the properties of cationic charge, polymer backbone and molecular weight optimization drive performance advantages. In general, traditional routes to achieve higher cationic substitution leads to less biodegradability. The present invention has found that while adding the biodegradability constraint in development, has resulted in unexpected innovation areas. For example, the low molecular weight cationic polyglucans of the present invention, have been shown to provide meaningful levels of coacervation, resulting in the enhanced deposition of scalp care actives.

### SUMMARY OF THE INVENTION

The present invention is directed to a personal care composition comprising from about 8 to about 20% of one or more surfactants; from about 0.01 to about 10% of scalp care active; from about 0.01 to about 5% of one or more cationic polymer; from about 0.1% to about 5% of a cationically modified poly alpha-1,6-glucan ether compound; and wherein the composition has an increase in deposition of the scalp care active when compared to a control composition with no cationically modified poly alpha-1,6-glucan ether compound.

### DETAILED DESCRIPTION OF THE INVENTION

Formulating the personal care composition with a surfactant system and a cationic poly alpha-1,6-glucan ether compound, as described herein, has been found to result in improved to provide wet conditioning as well as improve the deposition efficiency of actives.

All percentages and ratios used herein are by weight of the total composition, unless otherwise designated. All measurements are understood to be made at ambient conditions, where “ambient conditions” means conditions at about 25° C., under about one atmosphere of pressure, and at about 50% relative humidity, unless otherwise designated. All numeric ranges are inclusive of narrower ranges; delineated upper and lower range limits are combinable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

“Apply” or “application,” as used in reference to a composition, means to apply or spread the compositions of the present invention onto keratinous tissue such as the hair.

“Dermatologically acceptable” means that the compositions or components described are suitable for use in contact with human skin tissue without undue toxicity, incompatibility, instability, allergic response, and the like.

“Safe and effective amount” means an amount of a compound or composition sufficient to significantly induce a positive benefit.

“Sulfated surfactants” means surfactants that contain a sulfate moiety. Some non-limiting examples of sulfated surfactants are sodium lauryl sulfate, sodium laureth sulfate, ammonium lauryl sulfate, and ammonium laureth sulfate.

“Sulfate-free surfactant” refers to a surfactant that has no sulfate moieties.

While the specification concludes with claims particularly pointing out and distinctly claiming the invention, it is believed that the present invention will be better understood from the following description.

As used herein, the term “fluid” includes liquids and gels.

As used herein, the articles including “a” and “an” when used in a claim, are understood to mean one or more of what is claimed or described.

As used herein, “comprising” means that other steps and other ingredients which do not affect the end result can be added. This term encompasses the terms “consisting of” and “consisting essentially of”.

As used herein, “mixtures” is meant to include a simple combination of materials and any compounds that may result from their combination.

As used herein, “molecular weight” or “Molecular weight” refers to the weight average molecular weight unless otherwise stated. Molecular weight is measured using industry standard method, gel permeation chromatography (“GPC”).

Where amount ranges are given, these are to be understood as being the total amount of said ingredient in the composition, or where more than one species fall within the scope of the ingredient definition, the total amount of all ingredients fitting that definition, in the composition.

For example, if the composition comprises from 1% to 5% fatty alcohol, then a composition comprising 2% stearyl alcohol and 1% cetyl alcohol and no other fatty alcohol, would fall within this scope.

The amount of each particular ingredient or mixtures thereof described hereinafter can account for up to 100% (or 100%) of the total amount of the ingredient(s) in the personal care composition.

As used herein, “personal care compositions” includes products such as shampoos, shower gels, liquid hand cleansers, hair colorants, facial cleansers, and other surfactant-based liquid compositions

As used herein, the terms “include,” “includes,” and “including,” are meant to be non-limiting and are understood to mean “comprise,” “comprises,” and “comprising,” respectively.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Unless otherwise noted, all component or composition levels are in reference to the active portion of that component or composition, and are exclusive of impurities, for example, residual solvents or by-products, which may be present in commercially available sources of such components or compositions.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Scalp Care Active

The personal care composition of the present invention may contain a scalp care active. A scalp care active may be one material or a mixture selected from the groups consisting of hydroxy pyridones, such as octopirox (piroctone olamine), ciclopirox, rilopirox, and MEA-Hydroxyoctyloxypyridinone; azoles, such as climbazole, ketoconazole, itraconazole, econazole, and elubiol; kerolytic agents, such as salicylic acid and other hydroxy acids; strobilurins such as azoxystrobin and metal chelators such as 1,10-phenanthroline; polyvalent metal salts of pyrithione, non-limiting examples include zinc pyrithione (ZPT) and copper pyrithione; sulfur, and selenium sulfide.

The scalp care active may be present in an amount from about 0.01% to 10%, from about 0.1% to about 9%, from about 0.25% to 8%, and from about 0.5% to 6%. The scalp care active can be surfactant soluble and thus surfactant soluble scalp care active.

### Detersive Surfactant

### Surfactant System

The surfactant system may be present at 5% to 50% (e.g., 15% to 40% or 20%-35%), based on the weight of the composition. The surfactant system may be present at 8% to 20% based on the weight of the composition. The surfactant system includes an anionic detersive surfactant and at least one co-surfactant selected from non-ionic surfactants, amphoteric surfactants and zwitterionic surfactants.

### Anionic Surfactant

Some nonlimiting examples of anionic surfactants that may be suitable for use herein are alkyl sulfates; alkyl ether sulfates; acyl glycinates; acyl sarcosinates; acyl glutamates; acyl alaninates; sulfosuccinates, isethionates; sulfonates; sulfoacetates; glucose carboxylates; alkyl ether carboxylates; acyl taurates; sodium, ammonium or potassium salts of these; and combinations thereof. In some instances, the alkyl sulfate anionic surfactant can alkoxylated with an average degree of alkoxylation of less than 3.5 (e.g., 0.3 to 2.0 or 0.5 to 0.9), which is believed to help improve low temperature physical stability and suds mileage of the composition. Methods for determining degree of alkoxylation are known in the art, for example, as described in US 2023/0045856.

Examples of anionic sulfate surfactants include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine, lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate and sodium cocoyl isethionate. Sodium lauryl sulfate or sodium laureth sulfate may be particularly suitable.

Examples of sulfosuccinate surfactants include disodium N-octadecyl sulfosuccinate, disodium lauryl sulfosuccinate, diammonium lauryl sulfosuccinate, sodium lauryl sulfosuccinate, disodium laureth sulfosuccinate, tetrasodium N-(1,2-dicarboxyethyl)-N-octadecyl sulfosuccinnate, diamyl ester of sodium sulfosuccinic acid, dihexyl ester of sodium sulfosuccinic acid and dioctyl esters of sodium sulfosuccinic acid.

Examples of isethionate surfactants include sodium lauroyl methyl isethionate, sodium cocoyl isethionate, ammonium cocoyl isethionate, sodium hydrogenated cocoyl methyl isethionate, sodium lauroyl isethionate, sodium cocoyl methyl isethionate, sodium myristoyl isethionate, sodium oleoyl isethionate, sodium oleyl methyl isethionate, sodium palm kerneloyl isethionate and sodium stearoyl methyl isethionate.

Examples of sulfonates include alpha olefin sulfonates (e.g., C14-16 alpha olefin sulfonate), linear alkylbenzene sulfonates and sodium laurylglucosides hydroxypropylsulfonate.

Examples of sulfoacetates include sodium lauryl sulfoacetate and ammonium lauryl sulfoacetate.

Example of glucose carboxylates include sodium lauryl glucoside carboxylate, sodium cocoyl glucoside carboxylate and combinations thereof.

Non-limiting example of alkyl ether carboxylate can include sodium laureth-4 carboxylate, laureth-5 carboxylate, laureth-13 carboxylate, sodium C12-13 pareth-8 carboxylate and sodium C12-15 pareth-8 carboxylate.

Examples of acyl taurates include sodium methyl cocoyl taurate, sodium cocoyl taurate, sodium methyl lauroyl taurate, sodium lauroyl taurate and sodium methyl oleoyl taurate.

### Co-Surfactant

The surfactant system herein may include 5% to 50% of a co-surfactant, based on the weight of the surfactant system and/or 1% to 15% (e.g., 2-10%, 3-9%, 4-8%, or even 5-7%), based on the weight of the composition. The amount of co-surfactant in the composition can be important and should be tailored to balance solubility and/or viscosity building with cleaning and/or conditioning benefit. For example, too much amphoteric co-surfactant can make the surfactant system less salt tolerant and may impede the ability of the surfactant system to form a suitable coacervate upon dilution with water. This can be especially problematic when the composition contains a cationic polymer because the lowered salt tolerance of the surfactant system may cause the cationic polymer to precipitate out. The co-surfactant may be present at a weight ratio of detersive surfactant to co-surfactant of 12:1 to 3:10 (6:1 to 3:10, 4:1 to 1:3, or even 2:1 to 1:2).

Some non-limiting examples of amphoteric and zwitterionic surfactants include derivatives of aliphatic secondary and tertiary amines in which one of the aliphatic substituents contains from 8 to 18 carbon atoms and one aliphatic substituent contains an anionic group such as a carboxy, sulfonate, phosphate, or phosphonate group. Zwitterionic surfactants are surfactants whose polar functional group has two permanent charges that do not change with changing pH. Amphoteric surfactants have polar functional groups whose charge depends on the pH of the solution and can exhibit different charges as the pH changes from acid to neutral to basic, ranging from cationic to zwitterionic and potentially even to anionic. Some non-limiting examples of zwitterionic surfactants include amidosulfobetaines, hydroxysultaines, amidopropyl hydroxysultaines, and combinations thereof. Some non-limiting examples of amphoteric surfactants include amphoacetates, amphodiacetates, betaines, amidobetaines (e.g., cocamidopropyl betaine and lauramidopropyl betaine), propionates, hydroxysultaines, and combinations thereof.

Some non-limiting examples of non-ionic surfactants include glyceryl esters of alkanoic acids, polyglyceryl esters of alkanoic acids, propylene glycol esters of alkanoic acids, sorbitol esters of alkanoic acids, alkanolamides, alkoxylated amides, alkyl glycosides, alkyl polyglucosides acyl glucamides, amine oxides and combinations thereof. Some particularly suitable examples of non-ionic surfactants include cocamide, cocamide MEA, PPG-2 cocamide, PPG-2 hydroxyethyl cocamide, PPG-2 hydroxyethyl isostearamide, lauroyl/myristoyl methyl glucamide, capryloyl/caproyl methyl glucamide, cocoyl methyl glucamide, decyl glucoside, coco-glucoside, lauryl glucoside, lauramine oxide, cocamine oxide and combinations thereof.

More specific examples of the optional co-surfactants described above are disclosed in US 2019/0105246, US 2018/0098923, U.S. Pat. No. 9,271,908, WO 2020/016097, and McCutcheon's Emulsifiers and Detergents, 2019, MC Publishing Co.

### Cationic Polymers

The personal care composition also comprises a cationic polymer. These cationic polymers can include at least one of (a) a cationic guar polymer, (b) a cationic non-guar galactomannan polymer, (c) a cationic tapioca polymer, (d) a cationic copolymer of acrylamide monomers and cationic monomers, and/or (e) a synthetic, non-crosslinked, cationic polymer, which may or may not form lyotropic liquid crystals upon combination with the detersive surfactant (f) a cationic cellulose polymer. Additionally, the cationic polymer can be a mixture of cationic polymers.

The personal care composition may comprise a cationic guar polymer, which is a cationically substituted galactomannan (guar) gum derivatives. Guar gum for use in preparing these guar gum derivatives is typically obtained as a naturally occurring material from the seeds of the guar plant. The guar molecule itself is a straight chain mannan, which is branched at regular intervals with single membered galactose units on alternative mannose units. The mannose units are linked to each other by means of β(1-4) glycosidic linkages. The galactose branching arises by way of an α(1-6) linkage. Cationic derivatives of the guar gums are obtained by reaction between the hydroxyl groups of the polygalactomannan and reactive quaternary ammonium compounds. The degree of substitution of the cationic groups onto the guar structure should be sufficient to provide the requisite cationic charge density described above.

In the present invention, the cationic polymer, may be, including but not limited, to a cationic guar polymer, has a weight average Molecular weight of less than 2.2 million g/mol, or from about 150 thousand to about 2.2 million g/mol, or from about 200 thousand to about 2.2 million g/mol, or from about 250 thousand to about 2.5 million g/mol, or from about 300 thousand to about 1.2 million g/mol, or from about 700,000 thousand to about 1 million g/mol. Further, the cationic guar polymer may have a charge density of from about 0.2 to about 2.2 meq/g, or from about 0.3 to about 2.0 meq/g, or from about 0.4 to about 1.8 meq/g; or from about 0.5 meq/g to about 1.8 meq/g.

The cationic guar polymer may have a weight average Molecular weight of less than about 1.5 million g/mol, and has a charge density of from about 0.1 meq/g to about 2.5 meq/g. The cationic guar polymer may have a weight average molecular weight of less than 900 thousand g/mol, or from about 150 thousand to about 800 thousand g/mol, or from about 200 thousand to about 700 thousand g/mol, or from about 300 thousand to about 700 thousand g/mol, or from about 400 thousand to about 600 thousand g/mol. from about 150 thousand to about 800 thousand g/mol, or from about 200 thousand to about 700 thousand g/mol, or from about 300 thousand to about 700 thousand g/mol, or from about 400 thousand to about 600 thousand g/mol. The cationic guar polymer may have a charge density of from about 0.2 to about 2.2 meq/g, or from about 0.3 to about 2.0 meq/g, or from about 0.4 to about 1.8 meq/g; or from about 0.5 meq/g to about 1.5 meq/g.

The cationic guar polymer may be formed from quaternary ammonium compounds. The quaternary ammonium compounds for forming the cationic guar polymer may conform to the general formula 1:

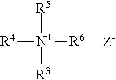

wherein where R³, R⁴ and R⁵ are methyl or ethyl groups; R⁶ is either an epoxyalkyl group of the general formula 2:

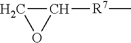

or R⁶ is a halohydrin group of the general formula 3:

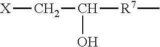

wherein R⁷ is a C₁ to C₃ alkylene; X is chlorine or bromine, and Z is an anion such as Cl—, Br—, I— or HSO₄—.

The cationic guar polymer may conform to the general formula 4:

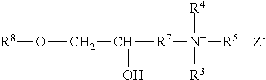

wherein R⁸ is guar gum; and wherein R⁴, R⁵, R⁶ and R⁷ are as defined above; and wherein Z is a halogen. The cationic guar polymer may conform to Formula 5:

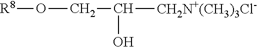

Suitable cationic guar polymers include cationic guar gum derivatives, such as guar hydroxypropyltrimonium chloride. The cationic guar polymer may be a guar hydroxypropyltrimonium chloride. Specific examples of guar hydroxypropyltrimonium chlorides include the Jaguar® series commercially available from Solvay, for example Jaguar® C-500, commercially available from Solvay. Jaguar® C-500 has a charge density of 0.8 meq/g and a molecular weight of 500,000 g/mol. Other suitable guar hydroxypropyltrimonium chloride are: guar hydroxypropyltrimonium chloride which has a charge density of about 1.3 meq/g and a molecular weight of about 500,000 g/mol and is available from Solvay as Jaguar® Optima. Other suitable guar hydroxypropyltrimonium chloride are: guar hydroxypropyltrimonium chloride which has a charge density of about 0.7 meq/g and a molecular weight of about 1,500,000 g/mol and is available from Solvay as Jaguar® Excel. Other suitable guar hydroxypropyltrimonium chloride are: guar hydroxypropyltrimonium chloride which has a charge density of about 1.1 meq/g and a molecular weight of about 500,000 g/mol and is available from ASI, a charge density of about 1.5 meq/g and a molecular weight of about 500,000 g/mole is available from ASI.

Other suitable guar hydroxypropyltrimonium chloride are: Hi-Care 1000, which has a charge density of about 0.7 meq/g and a Molecular weight of about 600,000 g/mole and is available from Solvay; N-Hance 3269 and N-Hance 3270, which have a charge density of about 0.7 meq/g and a molecular weight of about 425,000 g/mol and are available from ASI; N-Hance 3196, which has a charge density of about 0.8 meq/g and a molecular weight of about 1,100,000 g/mol and is available from ASI. AquaCat CG518 has a charge density of about 0.9 meq/g and a Molecular weight of about 50,000 g/mol and is available from ASI. BF-13, which is a borate (boron) free guar of charge density of about 1.1 meq/g and molecular weight of about 800,000 and BF-17, which is a borate (boron) free guar of charge density of about 1.5 meq/g and M. Wt. of about 800,000 both available from ASI.

The personal care compositions of the present invention may comprise a galactomannan polymer derivative having a mannose to galactose ratio of greater than 2:1 on a monomer to monomer basis, the galactomannan polymer derivative selected from the group consisting of a cationic galactomannan polymer derivative and an amphoteric galactomannan polymer derivative having a net positive charge. As used herein, the term “cationic galactomannan” refers to a galactomannan polymer to which a cationic group is added. The term “amphoteric galactomannan” refers to a galactomannan polymer to which a cationic group and an anionic group are added such that the polymer has a net positive charge.

Galactomannan polymers are present in the endosperm of seeds of the Leguminosae family. Galactomannan polymers are made up of a combination of mannose monomers and galactose monomers. The galactomannan molecule is a straight chain mannan branched at regular intervals with single membered galactose units on specific mannose units. The mannose units are linked to each other by means of β (1-4) glycosidic linkages. The galactose branching arises by way of an a (1-6) linkage. The ratio of mannose monomers to galactose monomers varies according to the species of the plant and also is affected by climate. Non Guar Galactomannan polymer derivatives of the present invention have a ratio of mannose to galactose of greater than 2:1 on a monomer to monomer basis. Suitable ratios of mannose to galactose can be greater than about 3:1, and the ratio of mannose to galactose can be greater than about 4:1. Analysis of mannose to galactose ratios is well known in the art and is typically based on the measurement of the galactose content.

The gum for use in preparing the non-guar galactomannan polymer derivatives is typically obtained as naturally occurring material such as seeds or beans from plants. Examples of various non-guar galactomannan polymers include but are not limited to Tara gum (3 parts mannose/1 part galactose), Locust bean or Carob (4 parts mannose/1 part galactose), and Cassia gum (5 parts mannose/1 part galactose).

The non-guar galactomannan polymer derivatives may have a M. Wt. from about 1,000 to about 10,000,000, and/or from about 5,000 to about 3,000,000.

The personal care compositions of the invention can also include galactomannan polymer derivatives which have a cationic charge density from about 0.5 meq/g to about 7 meq/g. The galactomannan polymer derivatives may have a cationic charge density from about 1 meq/g to about 5 meq/g. The degree of substitution of the cationic groups onto the galactomannan structure should be sufficient to provide the requisite cationic charge density.

The galactomannan polymer derivative can be a cationic derivative of the non-guar galactomannan polymer, which is obtained by reaction between the hydroxyl groups of the polygalactomannan polymer and reactive quaternary ammonium compounds. Suitable quaternary ammonium compounds for use in forming the cationic galactomannan polymer derivatives include those conforming to the general formulas 1-5, as defined above.

Cationic non-guar galactomannan polymer derivatives formed from the reagents described above are represented by the general formula 6:

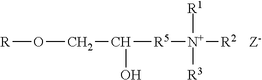

wherein R is the gum. The cationic galactomannan derivative can be a gum hydroxypropyltrimethylammonium chloride, which can be more specifically represented by the general formula 7:

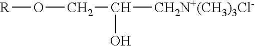

Alternatively the galactomannan polymer derivative can be an amphoteric galactomannan polymer derivative having a net positive charge, obtained when the cationic galactomannan polymer derivative further comprises an anionic group.

The cationic non-guar galactomannan can have a ratio of mannose to galactose is greater than about 4:1, a molecular weight of about 1,000 g/mol to about 10,000,000 g/mol, and/or from about 50,000 g/mol to about 1,000,000 g/mol, and/or from about 100,000 g/mol to about 900,000 g/mol, and/or from about 150,000 g/mol to about 400,000 g/mol and a cationic charge density from about 1 meq/g to about 5 meq/g, and/or from 2 meq/g to about 4 meq/g and can be derived from a cassia plant.

The personal care compositions can comprise water-soluble cationically modified starch polymers. As used herein, the term “cationically modified starch” refers to a starch to which a cationic group is added prior to degradation of the starch to a smaller molecular weight, or wherein a cationic group is added after modification of the starch to achieve a desired molecular weight. The definition of the term “cationically modified starch” also includes amphoterically modified starch. The term “amphoterically modified starch” refers to a starch hydrolysate to which a cationic group and an anionic group are added.

The cationically modified starch polymers disclosed herein have a percent of bound nitrogen of from about 0.5% to about 4%.

The cationically modified starch polymers for use in the personal care compositions can have a molecular weight about 850,000 g/mol to about 1,500,000 g/mol and/or from about 900,000 g/mol to about 1,500,000 g/mol.

The personal care compositions can include cationically modified starch polymers which have a charge density of from about 0.2 meq/g to about 5 meq/g, and/or from about 0.2 meq/g to about 2 meq/g. The chemical modification to obtain such a charge density includes, but is not limited to, the addition of amino and/or ammonium groups into the starch molecules. Non-limiting examples of these ammonium groups may include substituents such as hydroxypropyl trimmonium chloride, trimethylhydroxypropyl ammonium chloride, dimethylstearylhydroxypropyl ammonium chloride, and dimethyldodecylhydroxypropyl ammonium chloride. See Solarek, D. B., Cationic Starches in Modified Starches: Properties and Uses, Wurzburg, O. B., Ed., CRC Press, Inc., Boca Raton, Fla. 1986, pp 113-125. The cationic groups may be added to the starch prior to degradation to a smaller molecular weight or the cationic groups may be added after such modification.

The cationically modified starch polymers generally have a degree of substitution of a cationic group from about 0.2 to about 2.5. As used herein, the “degree of substitution” of the cationically modified starch polymers is an average measure of the number of hydroxyl groups on each anhydroglucose unit which is derivatized by substituent groups. Since each anhydroglucose unit has three potential hydroxyl groups available for substitution, the maximum possible degree of substitution is 3. The degree of substitution is expressed as the number of moles of substituent groups per mole of anhydroglucose unit, on a molar average basis. The degree of substitution may be determined using proton nuclear magnetic resonance spectroscopy (“.sup.1H NMR”) methods well known in the art. Suitable.sup.1H NMR techniques include those described in “Observation on NMR Spectra of Starches in Dimethyl Sulfoxide, Iodine-Complexing, and Solvating in Water-Dimethyl Sulfoxide”, Qin-Ji Peng and Arthur S. Perlin, Carbohydrate Research, 160 (1987), 57-72; and “An Approach to the Structural Analysis of Oligosaccharides by NMR Spectroscopy”, J. Howard Bradbury and J. Grant Collins, Carbohydrate Research, 71, (1979), 15-25.

The source of starch before chemical modification can be chosen from a variety of sources such as tubers, legumes, cereal, and grains. Non-limiting examples of this source starch may include corn starch, wheat starch, rice starch, waxy corn starch, oat starch, cassava starch, waxy barley, waxy rice starch, glutenous rice starch, sweet rice starch, amioca, potato starch, tapioca starch, oat starch, sago starch, sweet rice, or mixtures thereof.

The cationically modified starch polymers can be selected from degraded cationic maize starch, cationic tapioca, cationic potato starch, and mixtures thereof. Alternatively, the cationically modified starch polymers are cationic corn starch and cationic tapioca.

The starch, prior to degradation or after modification to a smaller molecular weight, may comprise one or more additional modifications. For example, these modifications may include cross-linking, stabilization reactions, phosphorylations, and hydrolyzations. Stabilization reactions may include alkylation and esterification.

The cationically modified starch polymers may be incorporated into the composition in the form of hydrolyzed starch (e.g., acid, enzyme, or alkaline degradation), oxidized starch (e.g., peroxide, peracid, hypochlorite, alkaline, or any other oxidizing agent), physically/mechanically degraded starch (e.g., via the thermo-mechanical energy input of the processing equipment), or combinations thereof.

An optimal form of the starch is one which is readily soluble in water and forms a substantially clear (% Transmittance of about 80 at 600 nm) solution in water. The transparency of the composition is measured by Ultra-Violet/Visible (UV/VIS) spectrophotometry, which determines the absorption or transmission of UV/VIS light by a sample, using a Gretag Macbeth Colorimeter Color i 5 according to the related instructions. A light wavelength of 600 nm has been shown to be adequate for characterizing the degree of clarity of cosmetic compositions.

Suitable cationically modified starch for use in personal care compositions are available from known starch suppliers. Also suitable for use in personal care compositions are nonionic modified starch that can be further derivatized to a cationically modified starch as is known in the art. Other suitable modified starch starting materials may be quaternized, as is known in the art, to produce the cationically modified starch polymer suitable for use in personal care compositions.

Starch Degradation Procedure: a starch slurry can be prepared by mixing granular starch in water. The temperature is raised to about 35° C. An aqueous solution of potassium permanganate is then added at a concentration of about 50 ppm based on starch. The pH is raised to about 11.5 with sodium hydroxide and the slurry is stirred sufficiently to prevent settling of the starch. Then, about a 30% solution of hydrogen peroxide diluted in water is added to a level of about 1% of peroxide based on starch. The pH of about 11.5 is then restored by adding additional sodium hydroxide. The reaction is completed over about a 1 to about 20 hour period. The mixture is then neutralized with dilute hydrochloric acid. The degraded starch is recovered by filtration followed by washing and drying.

The personal care composition can comprise a cationic copolymer of an acrylamide monomer and a cationic monomer, wherein the copolymer has a charge density of from about 1.0 meq/g to about 3.0 meq/g. The cationic copolymer can be a synthetic cationic copolymer of acrylamide monomers and cationic monomers.

The cationic copolymer can comprise:
(i) an acrylamide monomer of the following Formula AM:

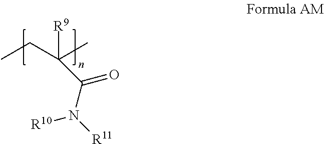

where R⁹ is H or C₁₋₄ alkyl; and R¹⁰ and R¹¹ are independently selected from the group consisting of H, C₁₋₄ alkyl, CH₂OCH₃, CH₂OCH₂CH(CH₃)₂, and phenyl, or together are C₃₋₆cycloalkyl; and
(ii) a cationic monomer conforming to Formula CM:

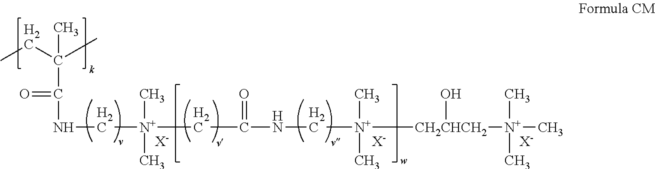

where k=1, each of v, v′, and v″ is independently an integer of from 1 to 6, w is zero or an integer of from 1 to 10, and X⁻ is an anion.

The cationic monomer can conform to Formula CM and where k=1, v=3 and w=0, z=1 and X⁻ is Cl⁻ to form the following structure:

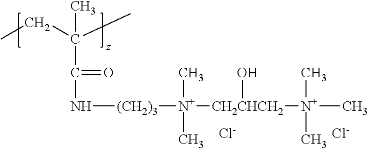

The above structure may be referred to as diquat. Alternatively, the cationic monomer can conform to Formula CM and wherein v and v″ are each 3, v′=1, w=1, y=1 and X⁻ is Cl⁻, such as:

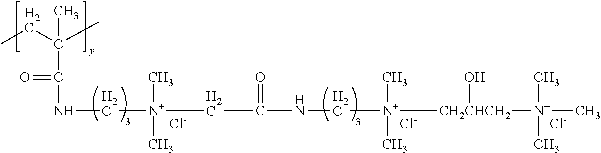

The above structure may be referred to as triquat.

Suitable acrylamide monomer include, but are not limited to, either acrylamide or methacrylamide.

The cationic copolymer (b) can be AM:TRIQUAT which is a copolymer of acrylamide and 1,3-Propanediaminium,N-[2-[[[dimethyl[3-[(2-methyl-1-oxo-2-propenyl)amino]propyl]ammonio]acetyl]amino]ethyl]2-hydroxy-N,N,N′,N′,N′-pentamethyl-, trichloride. AM:TRIQUAT is also known as polyquaternium 76 (PQ76). AM:TRIQUAT may have a charge density of 1.6 meq/g and a molecular weight of 1.1 million g/mol.

Further, the cationic copolymer may be of an acrylamide monomer and a cationic monomer, wherein the cationic monomer is selected from the group consisting of: dimethylaminoethyl (meth)acrylate, dimethylaminopropyl (meth)acrylate, ditertiobutylaminoethyl (meth)acrylate, dimethylaminomethyl (meth)acrylamide, dimethylaminopropyl (meth)acrylamide; ethylenimine, vinylamine, 2-vinylpyridine, 4-vinylpyridine; trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, diallyldimethyl ammonium chloride, and mixtures thereof.

The cationic copolymer can comprise a cationic monomer selected from the group consisting of: cationic monomers include trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, and mixtures thereof.

The cationic copolymer can be water-soluble. The cationic copolymer is formed from (1) copolymers of (meth)acrylamide and cationic monomers based on (meth)acrylamide, and/or hydrolysis-stable cationic monomers, (2) terpolymers of (meth)acrylamide, monomers based on cationic (meth)acrylic acid esters, and monomers based on (meth)acrylamide, and/or hydrolysis-stable cationic monomers. Monomers based on cationic (meth)acrylic acid esters may be cationized esters of the (meth)acrylic acid containing a quaternized N atom. The cationized esters of the (meth)acrylic acid containing a quaternized N atom may be quaternized dialkylaminoalkyl (meth)acrylates with C1 to C₃ in the alkyl and alkylene groups. Suitable cationized esters of the (meth)acrylic acid containing a quaternized N atom can be selected from the group consisting of: ammonium salts of dimethylaminomethyl (meth)acrylate, dimethylaminoethyl (meth)acrylate, dimethylaminopropyl (meth)acrylate, diethylaminomethyl (meth)acrylate, diethylaminoethyl (meth)acrylate; and diethylaminopropyl (meth)acrylate quaternized with methyl chloride. The cationized esters of the (meth)acrylic acid containing a quaternized N atom may be dimethylaminoethyl acrylate, which is quaternized with an alkyl halide, or with methyl chloride or benzyl chloride or dimethyl sulfate (ADAME-Quat). the cationic monomer when based on (meth)acrylamides can be quaternized dialkylaminoalkyl(meth)acrylamides with C1 to C₃ in the alkyl and alkylene groups, or dimethylaminopropylacrylamide, which is quaternized with an alkyl halide, or methyl chloride or benzyl chloride or dimethyl sulfate.

Suitable cationic monomer based on a (meth)acrylamide include quaternized dialkylaminoalkyl(meth)acrylamide with C1 to C₃ in the alkyl and alkylene groups. The cationic monomer based on a (meth)acrylamide can be dimethylaminopropylacrylamide, which is quaternized with an alkyl halide, especially methyl chloride or benzyl chloride or dimethyl sulfate.

The cationic monomer can be a hydrolysis-stable cationic monomer. Hydrolysis-stable cationic monomers can be, in addition to a dialkylaminoalkyl(meth)acrylamide, all monomers that can be regarded as stable to the OECD hydrolysis test. The cationic monomer can be hydrolysis-stable and the hydrolysis-stable cationic monomer can be selected from the group consisting of: diallyldimethylammonium chloride and water-soluble, cationic styrene derivatives.

The cationic copolymer can be a terpolymer of acrylamide, 2-dimethylammoniumethyl (meth)acrylate quaternized with methyl chloride (ADAME-Q) and 3-dimethylammoniumpropyl(meth)acrylamide quaternized with methyl chloride (DIMAPA-Q). The cationic copolymer can be formed from acrylamide and acrylamidopropyltrimethylammonium chloride, wherein the acrylamidopropyltrimethylammonium chloride has a charge density of from about 1.0 meq/g to about 3.0 meq/g.

The cationic copolymer can have a charge density of from about 1.1 meq/g to about 2.5 meq/g, or from about 1.1 meq/g to about 2.3 meq/g, or from about 1.2 meq/g to about 2.2 meq/g, or from about 1.2 meq/g to about 2.1 meq/g, or from about 1.3 meq/g to about 2.0 meq/g, or from about 1.3 meq/g to about 1.9 meq/g.

The cationic copolymer can have a molecular weight from about 100 thousand g/mol to about 1.5 million g/mol, or from about 300 thousand g/mol to about 1.5 million g/mol, or from about 500 thousand g/mol to about 1.5 million g/mol, or from about 700 thousand g/mol to about 1.0 million g/mol, or from about 900 thousand g/mol to about 1.2 million g/mol.

The cationic copolymer can be a trimethylammoniopropylmethacrylamide chloride-N-Acrylamide copolymer, which is also known as AM:MAPTAC. AM:MAPTAC may have a charge density of about 1.3 meq/g and a molecular weight of about 1.1 million g/mol. The cationic copolymer can be AM:ATPAC. AM:ATPAC can have a charge density of about 1.8 meq/g and a molecular weight of about 1.1 million g/mol.

### (a) Cationic Synthetic Polymers

The personal care composition can comprise a cationic synthetic polymer that may be formed from
i) one or more cationic monomer units, and optionally
ii) one or more monomer units bearing a negative charge, and/or
iii) a nonionic monomer,
   wherein the subsequent charge of the copolymer is positive. The ratio of the three types of monomers is given by “m”, “p” and “q” where “m” is the number of cationic monomers, “p” is the number of monomers bearing a negative charge and “q” is the number of nonionic monomers

The cationic polymers can be water soluble or dispersible, non-crosslinked, and synthetic cationic polymers having the following structure:

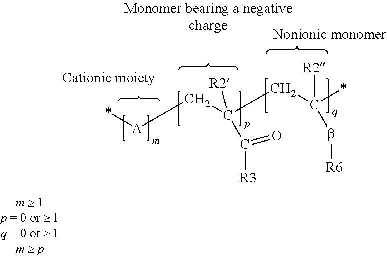

where A, may be one or more of the following cationic moieties:

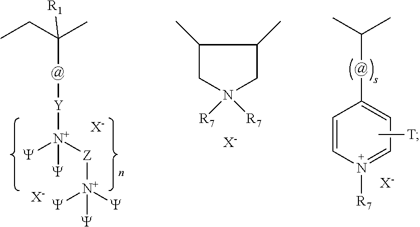

where @=amido, alkylamido, ester, ether, alkyl or alkylaryl;
where Y=C1-C22 alkyl, alkoxy, alkylidene, alkyl or aryloxy;
where ψ1=C1-C22 alkyl, alkyloxy, alkyl aryl or alkyl arylox;
where Z=C1-C22 alkyl, alkyloxy, aryl or aryloxy;
where R1=H, C₁-C₄ linear or branched alkyl;
where s=0 or 1, n=0 or ≥1;
where T and R7=C1-C22 alkyl; and
where X—=halogen, hydroxide, alkoxide, sulfate or alkylsulfate.

Where the monomer bearing a negative charge is defined by R2′=H, C1-C4 linear or branched alkyl and R3 as:

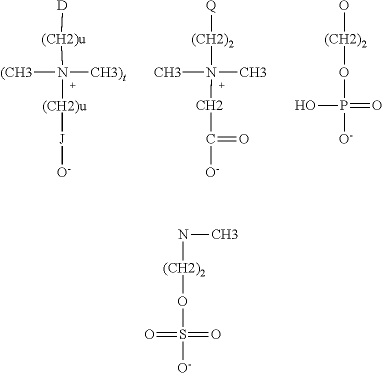

where D=O, N, or S;
where Q=NH₂ or O;
where u=1-6;
where t=0-1; and
where J=oxygenated functional group containing the following elements P, S, C.

Where the nonionic monomer is defined by R2″=H, C1-C4 linear or branched alkyl, R6=linear or branched alkyl, alkyl aryl, aryl oxy, alkyloxy, alkylaryl oxy and β is defined as

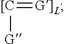

and
where G′ and G″ are, independently of one another, O, S or N—H and L=0 or 1.

Examples of cationic monomers include aminoalkyl (meth)acrylates, (meth)aminoalkyl (meth)acrylamides; monomers comprising at least one secondary, tertiary or quaternary amine function, or a heterocyclic group containing a nitrogen atom, vinylamine or ethylenimine; diallyldialkyl ammonium salts; their mixtures, their salts, and macromonomers deriving from therefrom.

Further examples of cationic monomers include dimethylaminoethyl (meth)acrylate, dimethylaminopropyl (meth)acrylate, ditertiobutylaminoethyl (meth)acrylate, dimethylaminomethyl (meth)acrylamide, dimethylaminopropyl (meth)acrylamide, ethylenimine, vinylamine, 2-vinylpyridine, 4-vinylpyridine, trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, diallyldimethyl ammonium chloride.

Suitable cationic monomers include those which comprise a quaternary ammonium group of formula —NR₃⁺, wherein R, which is identical or different, represents a hydrogen atom, an alkyl group comprising 1 to 10 carbon atoms, or a benzyl group, optionally carrying a hydroxyl group, and comprise an anion (counter-ion). Examples of anions are halides such as chlorides, bromides, sulphates, hydrosulphates, alkylsulphates (for example comprising 1 to 6 carbon atoms), phosphates, citrates, formates, and acetates.

Suitable cationic monomers include trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride.

Additional suitable cationic monomers include trimethyl ammonium propyl (meth)acrylamido chloride.

Examples of monomers bearing a negative charge include alpha ethylenically unsaturated monomers comprising a phosphate or phosphonate group, alpha ethylenically unsaturated monocarboxylic acids, monoalkylesters of alpha ethylenically unsaturated dicarboxylic acids, monoalkylamides of alpha ethylenically unsaturated dicarboxylic acids, alpha ethylenically unsaturated compounds comprising a sulphonic acid group, and salts of alpha ethylenically unsaturated compounds comprising a sulphonic acid group.

Suitable monomers with a negative charge include acrylic acid, methacrylic acid, vinyl sulphonic acid, salts of vinyl sulfonic acid, vinylbenzene sulphonic acid, salts of vinylbenzene sulphonic acid, alpha-acrylamidomethylpropanesulphonic acid, salts of alpha-acrylamidomethylpropanesulphonic acid, 2-sulphoethyl methacrylate, salts of 2-sulphoethyl methacrylate, acrylamido-2-methylpropanesulphonic acid (AMPS), salts of acrylamido-2-methylpropanesulphonic acid, and styrenesulphonate (SS).

Examples of nonionic monomers include vinyl acetate, amides of alpha ethylenically unsaturated carboxylic acids, esters of an alpha ethylenically unsaturated monocarboxylic acids with an hydrogenated or fluorinated alcohol, polyethylene oxide (meth)acrylate (i.e. polyethoxylated (meth)acrylic acid), monoalkylesters of alpha ethylenically unsaturated dicarboxylic acids, monoalkylamides of alpha ethylenically unsaturated dicarboxylic acids, vinyl nitriles, vinylamine amides, vinyl alcohol, vinyl pyrolidone, and vinyl aromatic compounds.

Suitable nonionic monomers include styrene, acrylamide, methacrylamide, acrylonitrile, methylacrylate, ethylacrylate, n-propylacrylate, n-butylacrylate, methylmethacrylate, ethylmethacrylate, n-propylmethacrylate, n-butylmethacrylate, 2-ethyl-hexyl acrylate, 2-ethyl-hexyl methacrylate, 2-hydroxyethylacrylate and 2-hydroxyethylmethacrylate.

The anionic counterion (X—) in association with the synthetic cationic polymers may be any known counterion so long as the polymers remain soluble or dispersible in water, in the personal care composition, or in a coacervate phase of the personal care composition, and so long as the counterions are physically and chemically compatible with the essential components of the personal care composition or do not otherwise unduly impair product performance, stability or aesthetics. Non limiting examples of such counterions include halides (e.g., chlorine, fluorine, bromine, iodine), sulfate and methylsulfate.

The cationic polymer described herein can aid in providing damaged hair, particularly chemically treated hair, with a surrogate hydrophobic F-layer. The microscopically thin F-layer provides natural weatherproofing, while helping to seal in moisture and prevent further damage. Chemical treatments damage the hair cuticle and strip away its protective F-layer. As the F-layer is stripped away, the hair becomes increasingly hydrophilic. It has been found that when lyotropic liquid crystals are applied to chemically treated hair, the hair becomes more hydrophobic and more virgin-like, in both look and feel. Without being limited to any theory, it is believed that the lyotropic liquid crystal complex creates a hydrophobic layer or film, which coats the hair fibers and protects the hair, much like the natural F-layer protects the hair. The hydrophobic layer returns the hair to a generally virgin-like, healthier state. Lyotropic liquid crystals are formed by combining the synthetic cationic polymers described herein with the aforementioned anionic detersive surfactant component of the personal care composition. The synthetic cationic polymer has a relatively high charge density. It should be noted that some synthetic polymers having a relatively high cationic charge density do not form lyotropic liquid crystals, primarily due to their abnormal linear charge densities. Such synthetic cationic polymers are described in WO 94/06403 to Reich et al. The synthetic polymers described herein can be formulated in a stable personal care composition that provides improved conditioning performance, with respect to damaged hair.

Cationic synthetic polymers that can form lyotropic liquid crystals may have a cationic charge density of from about 2 meq/gm to about 7 meq/gm, and/or from about 3 meq/gm to about 7 meq/gm, and/or from about 4 meq/gm to about 7 meq/gm. The cationic charge density may be about 6.2 meq/gm. The polymers also have a M. Wt. of from about 1,000 to about 5,000,000, and/or from about 10,000 to about 1,500,000, and/or from about 100,000 to about 1,500,000.

The cationic synthetic polymers that provide enhanced conditioning and deposition of benefit agents but do not necessarily form lyotropic liquid crystals may have a cationic charge density of from about 0.7 meq/gm to about 7 meq/gm, and/or from about 0.8 meq/gm to about 5 meq/gm, and/or from about 1.0 meq/gm to about 3 meq/gm. The polymers also have a M. Wt. of from about 1,000 to about 1,500,000, from about 10,000 to about 1,500,000, and from about 100,000 to about 1,500,000.

Suitable cationic cellulose polymers are salts of hydroxyethyl cellulose reacted with trimethyl ammonium substituted epoxide, referred to in the industry (CTFA) as Polyquaternium 10 and available from Dow/Amerchol Corp. (Edison, N.J., USA) in their Polymer LR, JR, and KG series of polymers. Non-limiting examples include: JR-400, JR-125, JR-30M, KG-30M, JP, LR-400 and mixtures thereof. Other suitable types of cationic cellulose include the polymeric quaternary ammonium salts of hydroxyethyl cellulose reacted with lauryl dimethyl ammonium-substituted epoxide referred to in the industry (CTFA) as Polyquaternium 24. These materials are available from Dow/Amerchol Corp. under the tradename Polymer LM-200. Other suitable types of cationic cellulose include the polymeric quaternary ammonium salts of hydroxyethyl cellulose reacted with lauryl dimethyl ammonium-substituted epoxide and trimethyl ammonium substituted epoxide referred to in the industry (CTFA) as Polyquaternium 67. These materials are available from Dow/Amerchol Corp. under the tradename SoftCAT Polymer SL-5, SoftCAT Polymer SL-30, Polymer SL-60, Polymer SL-100, Polymer SK-L, Polymer SK-M, Polymer SK-MH, and Polymer SK-H.

Suitable cationic cellulose polymers may have a cationic charge density of from about 0.5 meq/gm to about 2.5 meq/gm, and/or from about 0.6 meq/gm to about 2.2 meq/gm, and/or from about 0.6 meq/gm to about 2.0 meq/gm. Further, the cationic charge density may be about 1.9 meq/gm. The polymers also have a M. Wt. of from about 200,000 to about 3,000,000, and/or from about 300,000 to about 2,200,000, from about 1,000,000 to about 2,200,000 and/or from about 300,000 to about 1,500,000. The cationic cellulose polymer may have a cationic charge density of about 1.7 to about 2.1 meq/gm and a molecular weight of from about 1,000,000 to about 2,000,000.

In the present invention, wherein the one or more cationic polymer may have a charge density (CD) of from about 0.05 to about 7.0 meq/gm; the one or more cationic polymer may have a charge density (CD) of from about 0.7 to about 3.0 meq/gm. In the present invention, the personal care composition the one or more cationic polymer may have a molecular weight (MW) of from about 300,000 to about 2,600,000 g/mol; may have a molecular weight (MW) of from about 700,000 to about 2,600,000 g/mol. In the present invention the one or more cationic polymer may have a molecular weight (MW) of from about 700,000 to about 1,500,000 g/mol and a charge density (CD) of from about 0.5 to about 1.6 meq/gm.

The concentration of the cationic polymers ranges about 0.01% to about 5%, from about 0.08% to about 3%, from about 0.1% to about 2%, and/or from about 0.2% to about 1%, by weight of the personal care composition.

### Thickening Polymers

The personal care composition may comprise a thickening polymer to increase the viscosity of the composition. Suitable thickening polymers can be used. The personal care composition may comprise from about 0.25% to about 10% of a thickening polymer, from about 0.5% to about 8% of a thickening polymer, from about 1.0% to about 5% of a thickening polymer, and from about 1% to about 4% of a thickening polymer. The thickening polymer modifier may be a polyacrylate, polyacrylamide thickeners. The thickening polymer may be an anionic thickening polymer.

The personal care composition may comprise thickening polymers that are homopolymers based on acrylic acid, methacrylic acid or other related derivatives, non-limiting examples include polyacrylate, polymethacrylate, polyethylacrylate, and polyacrylamide.

The thickening polymers may be alkali swellable and hydrophobically-modified alkali swellable acrylic copolymers or methacrylate copolymers, non-limiting examples include acrylic acid/acrylonitrogens copolymer, acrylates/steareth-20 itaconate copolymer, acrylates/ceteth-20 itaconate copolymer, Acrylates/Aminoacrylates/C10-30 Alkyl PEG-20 Itaconate Copolymer, acrylates/aminoacrylates copolymer, acrylates/steareth-20 methacrylate copolymer, acrylates/beheneth-25 methacrylate copolymer, acrylates/steareth-20 methacrylate crosspolymer, acrylates/beheneth-25 methacrylate/HEMA crosspolymer, acrylates/vinyl neodecanoate crosspolymer, acrylates/vinyl isodecanoate crosspolymer, Acrylates/Palmeth-25 Acrylate Copolymer, Acrylic Acid/Acrylamidomethyl Propane Sulfonic Acid Copolymer, and acrylates/C10-C30 alkyl acrylate crosspolymer.

The thickening polymers may be soluble crosslinked acrylic polymers, a non-limiting example includes carbomers.

The thickening polymers may be an associative polymeric thickeners, non-limiting examples include: hydrophobically modified, alkali swellable emulsions, non-limiting examples include hydrophobically modified polypolyacrylates; hydrophobically modified polyacrylic acids, and hydrophobically modified polyacrylamides; hydrophobically modified polyethers wherein these materials may have a hydrophobe that can be selected from cetyl, stearyl, oleayl, and combinations thereof.

The thickening polymers may be used in combination with polyvinylpyrrolidone, crosslinked polyvinylpyrrolidone and derivatives. The thickening polymers may be combined with polyvinyalcohol and derivatives. The thickening polymers may be combined with polyethyleneimine and derivatives.

The thickening polymers may be combined with alginic acid based materials, non-limiting examples include sodium alginate, and alginic acid propylene glycol esters.

The thickening polymers may be used in combination with polyurethane polymers, non-limiting examples include: hydrophobically modified alkoxylated urethane polymers, non-limiting examples include PEG-150/decyl alcohol/SMDI copolymer, PEG-150/stearyl alcohol/SMDI copolymer, polyurethane-39.

The thickening polymers may be combined with an associative polymeric thickeners, non-limiting examples include: hydrophobically modified cellulose derivatives; and a hydrophilic portion of repeating ethylene oxide groups with repeat units from about 10 to about 300, from about 30 to about 200, from about 40 to about 150. Non-limiting examples of this class include PEG-120-methylglucose dioleate, PEG-(40 or 60) sorbitan tetraoleate, PEG-150 pentaerythrityl tetrastearate, PEG-55 propylene glycol oleate, PEG-150 distearate.

The thickening polymers may be combined with cellulose and derivatives, non-limiting examples include microcrystalline cellulose, carboxymethylcelluloses, hydroxyethylcellulose, hydroxypropylcellulose, hydroxypropyl methylcellulose, methylcellulose, ethyl cellulose; nitro cellulose; cellulose sulfate; cellulose powder; hydrophobically modified celluloses.

The thickening polymers may be combined with a guar and guar derivatives, non-limiting examples include hydroxypropyl guar, and hydroxypropyl guar hydroxypropyl trimonium chloride.

The thickening polymers may be combined with polyethylene oxide;polypropylene oxide; and POE-PPO copolymers.

The thickening polymers may be combined with polyalkylene glycols characterized by the general formula:

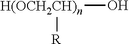

wherein R is hydrogen, methyl, or mixtures thereof, and further hydrogen, and n is an integer having an average from 2,000-180,000, or from 7,000-90,000, or from 7,000-45,000. Non-limiting examples of this class include PEG-7M, PEG-14M, PEG-23M, PEG-25M, PEG-45M, PEG-90M, or PEG-100M.

The thickening polymers may be combined with silicas, non-limiting examples include fumed silica, precipitated silica, and silicone-surface treated silica.

The thickening polymers may be combined with water-swellable clays, non-limiting examples include laponite, bentolite, montmorilonite, smectite, and hectonite.

The thickening polymers may be combined with gums, non-limiting examples include xanthan gum, guar gum, hydroxypropyl guar gum, Arabia gum, tragacanth, galactan, carob gum, karaya gum, and locust bean gum.

The thickening polymers may be combined with, dibenzylidene sorbitol, karaggenan, pectin, agar, quince seed (Cydonia oblonga Mill), starch (from rice, corn, potato, wheat, etc), starch-derivatives (e.g. carboxymethyl starch, methylhydroxypropyl starch), algae extracts, dextran, succinoglucan, and pulleran,

Non-limiting examples of thickening polymers include acrylamide/ammonium acrylate copolymer (and) polyisobutene (and) polysorbate 20; acrylamide/sodium acryloyldimethyl taurate copolymer/isohexadecane/polysorbate 80, ammonium acryloyldimethyltaurate/VP copolymer, Sodium Acrylate/Sodium Acryloyldimethyl Taurate Copolymer, acrylates copolymer, Acrylates Crosspolymer-4, Acrylates Crosspolymer-3, acrylates/beheneth-25 methacrylate copolymer, acrylates/C10-C30 alkyl acrylate crosspolymer, acrylates/steareth-20 itaconate copolymer, ammonium polyacrylate/Isohexadecane/PEG-40 castor oil; carbomer, sodium carbomer, crosslinked polyvinylpyrrolidone (PVP), polyacrylamide/C13-14 isoparaffin/laureth-7, polyacrylate 13/polyisobutene/polysorbate 20, polyacrylate crosspolymer-6, polyamide-3, polyquaternium-37 (and) hydrogenated polydecene (and) trideceth-6, Acrylamide/Sodium Acryloyldimethyltaurate/Acrylic Acid Copolymer, sodium acrylate/acryloyldimethyltaurate/dimethylacrylamide, crosspolymer (and) isohexadecane (and) polysorbate 60, sodium polyacrylate. Exemplary commercially-available thickening polymers include ACULYN™ 28, ACULYN™ 33, ACULYN™ 88, ACULYN™ 22, ACULYN™ Excel, Carbopol® Aqua SF-1, Carbopol® ETD 2020, Carbopol® Ultrez 20, Carbopol® Ultrez 21, Carbopol® Ultrez 10, Carbopol® Ultrez 30, Carbopol® 1342, Carbopol® Aqua SF-2 Polymer, Sepigel™ 305, Simulgel™ 600, Sepimax Zen, Carbopol® SMART 1000, Rheocare® TTA, Rheomer® SC-Plus, STRUCTURE® PLUS, Aristoflex® AVC, Stabylen 30 and combinations thereof.

### Gel Network

In the present invention, a gel network may be present. The gel network component of the present invention comprises at least one fatty amphiphile. As used herein, “fatty amphiphile” refers to a compound having a hydrophobic tail group as defined as an alkyl, alkenyl (containing up to 3 double bonds), alkyl aromatic, or branched alkyl group of C₁₂-C₇₀ length and a hydrophilic head group which does not make the compound water soluble, wherein the compound also has a net neutral charge at the pH of the shampoo composition.

The shampoo compositions of the present invention comprise fatty amphiphile as part of the pre-formed dispersed gel network phase in an amount from about 0.05% to about 14%, from about 0.5% to about 10%, and from about 1% to about 8%, by weight of the shampoo composition.

According to the present invention, suitable fatty amphiphiles, or suitable mixtures of two or more fatty amphiphiles, have a melting point of at least about 27° C. The melting point, as used herein, may be measured by a standard melting point method as described in U.S. Pharmacopeia, USP-NF General Chapter <741> “Melting range or temperature”. The melting point of a mixture of two or more materials is determined by mixing the two or more materials at a temperature above the respective melt points and then allowing the mixture to cool. If the resulting composite is a homogeneous solid below about 27° C., then the mixture has a suitable melting point for use in the present invention. A mixture of two or more fatty amphiphiles, wherein the mixture comprises at least one fatty amphiphile having an individual melting point of less than about 27° C., still is suitable for use in the present invention provided that the composite melting point of the mixture is at least about 27° C.

Suitable fatty amphiphiles of the present invention include fatty alcohols, alkoxylated fatty alcohols, fatty phenols, alkoxylated fatty phenols, fatty amides, alkyoxylated fatty amides, fatty amines, fatty alkylamidoalkylamines, fatty alkyoxyalted amines, fatty carbamates, fatty amine oxides, fatty acids, alkoxylated fatty acids, fatty diesters, fatty sorbitan esters, fatty sugar esters, methyl glucoside esters, fatty glycol esters, mono, di & tri glycerides, polyglycerine fatty esters, alkyl glyceryl ethers, propylene glycol fatty acid esters, cholesterol, ceramides, fatty silicone waxes, fatty glucose amides, and phospholipids and mixtures thereof.

The shampoo composition may comprise fatty alcohol gel networks. These gel networks are formed by combining fatty alcohols and surfactants in the ratio of from about 1:1 to about 40:1, from about 2:1 to about 20:1, and/or from about 3:1 to about 10:1. The formation of a gel network involves heating a dispersion of the fatty alcohol in water with the surfactant to a temperature above the melting point of the fatty alcohol. During the mixing process, the fatty alcohol melts, allowing the surfactant to partition into the fatty alcohol droplets. The surfactant brings water along with it into the fatty alcohol. This changes the isotropic fatty alcohol drops into liquid crystalline phase drops. When the mixture is cooled below the chain melt temperature, the liquid crystal phase is converted into a solid crystalline gel network. The gel network contributes a stabilizing benefit to cosmetic creams and hair conditioners. In addition, they deliver conditioned feel benefits for hair conditioners.

The fatty alcohol can be included in the fatty alcohol gel network at a level by weight of from about 0.05 wt % to about 14 wt %. For example, the fatty alcohol may be present in an amount ranging from about 1 wt % to about 10 wt %, and/or from about 6 wt % to about 8 wt %.

The fatty alcohols useful herein include those having from about 10 to about 40 carbon atoms, from about 12 to about 22 carbon atoms, from about 16 to about 22 carbon atoms, and/or about 16 to about 18 carbon atoms. These fatty alcohols can be straight or branched chain alcohols and can be saturated or unsaturated. Nonlimiting examples of fatty alcohols include cetyl alcohol, stearyl alcohol, behenyl alcohol, and mixtures thereof. Mixtures of cetyl and stearyl alcohol in a ratio of from about 20:80 to about 80:20 are suitable.

Gel network preparation: A vessel is charged with water and the water is heated to about 74° C. Cetyl alcohol, stearyl alcohol, and SLES surfactant are added to the heated water. After incorporation, the resulting mixture is passed through a heat exchanger where the mixture is cooled to about 35° C. Upon cooling, the fatty alcohols and surfactant crystallized to form a crystalline gel network. Table 1 provides the components and their respective amounts for an example gel network composition.

TABLE 1
Gel network components

| Ingredient | Wt. % |
|---|---|
| Water | 78.27% |
| Cetyl Alcohol | 4.18% |
| Stearyl Alcohol | 7.52% |
| Sodium laureth-3 sulfate (28% Active) | 10.00% |
| 5-Chloro-2-methyl-4-isothiazolin-3-one, Kathon CG | 0.03% |

### 1. Water Miscible Solvents

The carrier useful in the personal care composition may include water and water solutions of lower alkyl alcohols, polyhydric alcohols, ketones having from 3 to 4 carbons atoms, C1-C6 esters of C1-C6 alcohols, sulfoxides, amides, carbonate esters, ethoxylated and proposylated C1-C10 alcohols, lactones, pyrollidones, and mixtures thereof. Non-limited lower alkyl alcohol examples are monohydric alcohols having 1 to 6 carbons, such as ethanol and isopropanol. Non-limiting examples of polyhydric alcohols useful herein include propylene glycol, dipropylene glycol, butylenes glycol, hexylene glycol, glycerin, propane diol and mixtures thereof.

The personal care composition may comprise a hydrotrope/viscosity modifier which is an alkali metal or ammonium salt of a lower alkyl benzene sulphonate such as sodium xylene sulphonate, sodium cumene sulphonate or sodium toluene sulphonate.

The personal care composition may comprise silicone/PEG-8 silicone/PEG-9 silicone/PEG-n silicone/silicone ether (n could be another integer), non-limiting examples include PEG8-dimethicone A208) MW 855, PEG 8 Dimethicone D208 MW 2706.

Cationically Modified Poly alpha-1,6-glucan Ether Compound

The personal care composition comprises a cationically modified poly alpha-1,6-glucan ether compound. The cationically modified poly alpha-1,6-glucan ether compound may comprise a poly alpha-1,6-glucan substituted with at least one positively charged organic group, where the poly alpha-1,6-glucan comprises a backbone of glucose monomer units where at least 65% of the glucose monomer units are linked via alpha-1,6-glycosidic linkages, and where the poly alpha-1,6-glucan ether compound has a degree of substitution of about 0.001 to about 3; and is characterized by at least one of the following i-iv;
i) a weight average degree of polymerization of at least 5;
ii) a weight average molecular weight of from about 1000 to about 500,000 daltons;
iii) been derived from a poly alpha-1,6-glucan having a weight average molecular weight of from about 900 to about 450,000 daltons, determined prior to substitution with the least one positively charged organic group;
iv) a mixture thereof.

As used herein, the term “polysaccharide” means a polymeric carbohydrate molecule composed of long chains of monosaccharide units bound together by glycosidic linkages and on hydrolysis gives the constituent monosaccharides or oligosaccharides.

The term “polysaccharide derivative” as used herein means a chemically modified polysaccharide in which at least some of the hydroxyl groups of the glucose monomer units have been replaced with one or more ether groups. As used herein, the term “polysaccharide derivative” is used interchangeably with “poly alpha-1,6-glucan ether” and “poly alpha-1,6-glucan ether compound”.

As used herein, the term “cationically modified poly alpha-1,6-glucan ether compound” is used interchangeably with the INCI name “Alpha-Glucan Hydroxypropyltrimonium Chloride”.

The term “hydrophobic” refers to a molecule or substituent which is nonpolar and has little or no affinity for water, and which tends to repel water.

The term “hydrophilic” refers to a molecule or a substituent which is polar and has affinity to interact with polar solvents, particularly water, or with other polar groups. A hydrophilic molecule or substituent tends to attract water.

The “molecular weight” of a poly alpha-1,6-glucan or poly alpha-1,6-glucan ether can be represented as statistically averaged molecular mass distribution, i.e., as number-average molecular weight (Mₙ) or as weight-average molecular weight (M_{w}), both of which are generally given in units of Daltons (Da), i.e., in grams/mole. Alternatively, molecular weight can be represented as DPw (weight average degree of polymerization) or DPn (number average degree of polymerization). Various means are known in the art for calculating these molecular weights from techniques such as high-pressure liquid chromatography (HPLC), size exclusion chromatography (SEC), gel permeation chromatography (GPC), and gel filtration chromatography (GFC).

As used herein, “weight average molecular weight” or “M_{w}” is calculated as

M_{w}=ΣNᵢMᵢ²/ΣNᵢMᵢ; where Mᵢ is the molecular weight of an individual chain i and Nᵢ is the number of chains of that molecular weight. In addition to using SEC, the weight average molecular weight can be determined by other techniques such as static light scattering, mass spectrometry especially MALDI-TOF (matrix-assisted laser desorption/ionization time-of-flight), small angle X-ray or neutron scattering, and ultracentrifugation.

As used herein, “number average molecular weight” or “Mₙ” refers to the statistical average molecular weight of all the polymer chains in a sample. The number average molecular weight is calculated as Mₙ=ΣNᵢMᵢ/ΣNᵢ where Mᵢ is the molecular weight of a chain i and Nᵢ is the number of chains of that molecular weight. In addition to using SEC, the number average molecular weight of a polymer can be determined by various colligative methods such as vapor pressure osmometry or end-group determination by spectroscopic methods such as proton NMR, FTIR, or UV-vis.

As used herein, number average degree of polymerization (DPn) and weight average degree of polymerization (DPw) are calculated from the corresponding average molecular weights M_{w} or Mₙ by dividing by the molar mass of one monomer unit M₁. In the case of unsubstituted glucan polymer, M₁=162. In the case of a substituted glucan polymer, M₁=162+M_{f}×DoS, where M_{f} is the molar mass of the substituent group and DoS is the degree of substitution with respect to that substituent group (average number of substituted groups per one glucose unit).

Glucose carbon positions 1, 2, 3, 4, 5 and 6 as referred to herein are as known in the art and depicted in Structure I:

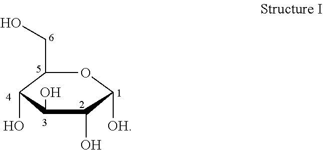

The terms “glycosidic linkage” and “glycosidic bond” are used interchangeably herein and refer to the type of covalent bond that joins a carbohydrate (sugar) molecule to another group such as another carbohydrate. The term “alpha-1,6-glucosidic linkage” as used herein refers to the covalent bond that joins alpha-D-glucose molecules to each other through carbons 1 and 6 on adjacent alpha-D-glucose rings. The term “alpha-1,3-glucosidic linkage” as used herein refers to the covalent bond that joins alpha-D-glucose molecules to each other through carbons 1 and 3 on adjacent alpha-D-glucose rings. The term “alpha-1,2-glucosidic linkage” as used herein refers to the covalent bond that joins alpha-D-glucose molecules to each other through carbons 1 and 2 on adjacent alpha-D-glucose rings. The term “alpha-1,4-glucosidic linkage” as used herein refers to the covalent bond that joins alpha-D-glucose molecules to each other through carbons 1 and 4 on adjacent alpha-D-glucose rings. Herein, “alpha-D-glucose” will be referred to as “glucose”.

The glycosidic linkage profile of a glucan, dextran, substituted glucan, or substituted dextran can be determined using any method known in the art. For example, a linkage profile can be determined using methods that use nuclear magnetic resonance (NMR) spectroscopy (e.g., ¹³C NMR or ¹H NMR). These and other methods that can be used are disclosed in Food Carbohydrates: Chemistry, Physical Properties, and Applications (S. W. Cui, Ed., Chapter 3, S. W. Cui, Structural Analysis of Polysaccharides, Taylor & Francis Group LLC, Boca Raton, FL, 2005), which is incorporated herein by reference.

The structure, molecular weight, and degree of substitution of a polysaccharide or polysaccharide derivative can be confirmed using various physiochemical analyses known in the art such as NMR spectroscopy and size exclusion chromatography (SEC).

The term “alkyl group”, as used herein, refers to linear, branched, aralkyl (such as benzyl), or cyclic (“cycloalkyl”) hydrocarbon groups containing no unsaturation. As used herein, the term “alkyl group” encompasses substituted alkyls, for example alkyl groups substituted with at least one hydroxyalkyl group or dihydroxy alkyl group, as well as alkyl groups containing one or more heteroatoms such as oxygen, sulfur, and/or nitrogen within the hydrocarbon chain.

As used herein, the term “aryl” means an aromatic carbocyclic group having a single ring (e.g., phenyl), multiple rings (e.g., biphenyl), or multiple condensed rings in which at least one is aromatic, (e.g., 1,2,3,4 tetrahydronaphthyl, naphthyl, anthryl, or phenanthryl), which is optionally mono, di, or trisubstituted with alkyl groups. By aryl is also meant heteroaryl groups where heteroaryl is defined as 5, 6, or 7 membered aromatic ring systems having at least one hetero atom selected from the group consisting of nitrogen, oxygen and sulfur. Examples of heteroaryl groups include pyridyl, pyrimidinyl, pyrrolyl, pyrazolyl, pyrazinyl, pyridazinyl, oxazolyl, furanyl, imidazole, quinolinyl, isoquinolinyl, thiazolyl, and thienyl, which can optionally be substituted with alkyl groups.

The poly alpha-1,6-glucan ether compound comprises a poly alpha-1,6-glucan substituted with at least one positively charged organic group, where the poly alpha-1,6-glucan comprises a backbone of glucose monomer units, where at least 65% of the glucose monomer units are linked via alpha-1,6-glycosidic linkages. The poly alpha-1,6-glucan ether compound may be characterized by (a) a weight average degree of polymerization of at least 5; (b) a weight average molecular weight of from about 1000 to about 500,000 daltons; and/or (c) having been derived from a poly alpha-1,6-glucan having a weight average molecular weight of from about 900 to about 450,000 daltons, determined prior to substitution with the least one positively charged organic group. The poly alpha-1,6-glucan ether compound may be characterized by a degree of substitution of about 0.001 to about 3.0. Optionally, at least 3%, may be from about 5% to about 50%, may be from about 5% to about 35%, of the backbone glucose monomer units have branches via alpha-1,2 and/or alpha-1,3-glycosidic linkages. These compounds, groups, and properties are described in more detail below.

The poly alpha-1,6-glucan ether compounds disclosed herein comprise poly alpha-1,6-glucan substituted with at least one positively charged organic group, wherein the organic group or groups are independently linked to the poly alpha-1,6-glucan polysaccharide backbone and/or to any branches, if present, through an ether (—O—) linkage. The at least one positively charged organic group can derivatize the poly alpha-1,6-glucan at the 2, 3, and/or 4 glucose carbon position(s) of a glucose monomer on the backbone of the glucan, and/or at the 1, 2, 3, 4, or 6 glucose carbon position(s) of a glucose monomer on a branch, if present. At unsubstituted positions a hydroxyl group is present in a glucose monomer.

The poly alpha-1,6-glucan ether compounds disclosed herein are referred to as “cationic” ether compounds due to the presence of one or more positively charged organic groups. The terms “positively charged organic group”, “positively charged ionic group”, and “cationic group” are used interchangeably herein. A positively charged group comprises a cation (a positively charged ion). Examples of positively charged groups include substituted ammonium groups, carbocation groups, and acyl cation groups.

The cationic poly alpha-1,6-glucan ether compounds disclosed herein comprise water-soluble poly alpha-1,6-glucan comprising a backbone of glucose monomer units wherein at least 65% of the glucose monomer units are linked via alpha-1,6-glycosidic linkages, and optionally at least 5% of the backbone glucose monomer units have branches via alpha-1,2 and/or alpha-1,3-glycosidic linkages. The poly alpha-1,6-glucan is substituted with positively charged organic groups on the polysaccharide backbone and/or on any branches which may be present, such that the poly alpha-1,6-glucan ether compound comprises unsubstituted and substituted alpha-D-glucose rings. The poly alpha-1,6-glucan may be randomly substituted with positively charged organic groups. As used herein, the term “randomly substituted” means the substituents on the glucose rings in the randomly substituted polysaccharide occur in a non-repeating or random fashion. That is, the substitution on a substituted glucose ring may be the same or different (i.e. the substituents, which may be the same or different, on different atoms in the glucose rings in the polysaccharide) from the substitution on a second substituted glucose ring in the polysaccharide, such that the overall substitution on the polymer has no pattern. Further, the substituted glucose rings may occur randomly within the polysaccharide (i.e., there is no pattern with the substituted and unsubstituted glucose rings within the polysaccharide).

Depending on reaction conditions and the specific substituent used to derivatize the poly alpha-1,6-glucan, the glucose monomers of the polymer backbone may be disproportionately substituted relative to the glucose monomers of any branches, including branches via alpha-1,2 and/or alpha-1,3 linkages, if present. The glucose monomers of the branches, including branches via alpha-1,2 and/or alpha-1,3 linkages, if present, may be disproportionately substituted relative to the glucose monomers of the polymer backbone. Depending on reaction conditions and the specific substituent used, substitution of the poly alpha-1,6-glucan may occur in a block manner.

Depending on reaction conditions and the specific substituent used to derivatize the poly alpha-1,6-glucan, it is possible that the hydroxyl groups at certain glucose carbon positions may be disproportionately substituted. For example, the hydroxyl at carbon position 6 for a branched unit may be more substituted than the hydroxyls at other carbon positions. The hydroxyl at carbon position 2, 3, or 4 may be more substituted than the hydroxyls at other carbon positions.

The poly alpha-1,6-glucan ether compounds disclosed herein contain positively charged organic groups and are of interest due to their solubility characteristics in water, which can be varied by appropriate selection of substituents and the degree of substitution. Compositions comprising the poly alpha-1,6-glucan ether compounds can be useful in personal care compositions. The poly alpha-1,6-glucan ether compound may have a DoS of about 0.001 to about 1.5 and a solubility of 0.1% by weight or higher in deionized water at 25° C. The poly alpha-1,6-glucan ether compound may have a DoS of about 0.05 to about 1.5 and a solubility of less than 0.1% by weight in pH 7 water at 25° C. Poly alpha-1,6-glucan ether compounds having a solubility of at least 0.1%, or at least 1%, or at least 10%, or at least 25%, or at least 50%, or at least 75%, or at least 90%, by weight, in deionized water at 25° C. may be for use in personal care compositions, due to ease of processing and/or increased solubility in aqueous end-use conditions.

The cationic poly alpha-1,6-glucan ether compounds disclosed herein can be comprised in a personal care composition in an effective amount, for example an amount that provides easy rinsing and improved solution feel.

The personal care composition may comprise from 0.01% to 5%, or from 0.05% to 3%, from 0.1% to 5%, or from 0.1% to 2%, or from 0.25% to 1.0%, by weight of the composition, of the poly alpha-1,6-glucan ether compound.

The poly alpha-1,6-glucan ether compounds of the present disclosure comprise a substituted poly alpha-1,6-glucan, and are typically made from a poly alpha-1,6-glucan starting material. The terms “poly alpha-1,6-glucan” and “dextran” are used interchangeably herein. Dextrans represent a family of complex, branched alpha-glucans generally comprising chains of alpha-1,6-linked glucose monomers, with periodic side chains (branches) linked to the straight chains by alpha-1,3-linkage (Joan et al., Macromolecules 33:5730-5739) or alpha-1,2-linkage. Production of dextrans is typically done through fermentation of sucrose with bacteria (e.g., Leuconostoc or Streptococcus species), where sucrose serves as the source of glucose for dextran polymerization (Naessens et al., J. Chem. Technol. Biotechnol. 80:845-860; Sarwat et al., Int. J. Biol. Sci. 4:379-386; Onilude et al., Int. Food Res. J. 20:1645-1651). Poly alpha-1,6-glucan can be prepared using glucosyltransferases such as (but not limited to) GTF1729, GTF1428, GTF5604, GTF6831, GTF8845, GTF0088, and GTF8117 as described in WO2015/183714 and WO2017/091533, both of which are incorporated herein by reference.

The cationic poly alpha-1,6-glucan ether compound may comprise a backbone of glucose monomer units wherein greater than or equal to 40% of the glucose monomer units are linked via alpha-1,6-glycosodic linkages, for example greater than or equal to 40%, 45%, 50%, 55%, 60%, 65%, 70%, 75%, 80%, 90%, or 95% of the glucose monomer units. The backbone of the cationic poly alpha-1,6-glucan ether compound can comprise at least 3%, 5%, 10%, 15%, 20%, 25%, 30%, 35%, 40%, 45%, 50%, 55%, or 60% glucose monomer units which are linked via alpha-1,2, alpha-1,3, and/or alpha-1,4 glycosidic linkages. The cationic poly alpha-1,6-glucan ether compound may comprise a backbone of glucose monomer units wherein at least 65% of the glucose monomer units are linked via alpha-1,6-glycosidic linkages. The cationic poly alpha-1,6-glucan ether compound may comprise a backbone of glucose monomer units wherein at least 70% of the glucose monomer units are linked via alpha-1,6-glycosidic linkages. The cationic poly alpha-1,6-glucan ether compound may comprise a backbone of glucose monomer units wherein at least 80% of the glucose monomer units are linked via alpha-1,6-glycosidic linkages. The cationic poly alpha-1,6-glucan ether compound may comprise a backbone of glucose monomer units wherein at least 90% of the glucose monomer units are linked via alpha-1,6-glycosidic linkages. The cationic poly alpha-1,6-glucan ether compound may comprise a backbone of glucose monomer units wherein at least 95% of the glucose monomer units are linked via alpha-1,6-glycosidic linkages. The cationic poly alpha-1,6-glucan ether compound may comprise a backbone of glucose monomer units wherein at least 99.5% of the glucose monomer units are linked via alpha-1,6-glycosidic linkages. The poly alpha-1,6-glucan ether compound may be predominantly linear.

Dextran “long chains” can comprise “substantially (or mostly) alpha-1,6-glucosidic linkages”, meaning that they can have at least about 98.0% alpha-1,6-glucosidic linkages in some aspects. Dextran herein can comprise a “branching structure” (branched structure) in some aspects. It is contemplated that in this structure, long chains branch from other long chains, likely in an iterative manner (e.g., a long chain can be a branch from another long chain, which in turn can itself be a branch from another long chain, and so on). It is contemplated that long chains in this structure can be “similar in length”, meaning that the length (e.g., measured by DP/degree of polymerization) of at least 70% of all the long chains in a branching structure is within plus/minus 30% of the mean length of all the long chains of the branching structure.

Dextran may further comprise “short chains” branching from the polysaccharide backbone, the branches typically being one to three glucose monomers in length, and typically comprising less than about 10% of all the glucose monomers of a dextran polymer. Such short chains typically comprise alpha-1,2-, alpha-1,3-, and/or alpha-1,4-glucosidic linkages (it is understood that there can also be a small percentage of such non-alpha-1,6 linkages in long chains in some aspects). The amount of alpha-1,2-branching or alpha-1,3-branching can be determined by NMR methods, as disclosed in the Test Methods.

Dextran can be produced enzymatically prior to being modified with alpha-1,2 or alpha-1,3 branches. The dextran may be synthesized using a dextransucrase and/or methodology as disclosed in WO 2015/183714 or WO 2017/091533 or published application US 2018/0282385, which are all incorporated herein by reference. The dextransucrase identified as GTF8117, GTF6831, or GTF5604 in these references can be used, if desired (or any dextransucrase comprising an amino acid sequence that is at least about 90%, 91%, 92%, 93%, 94%, 95%, 96%, 97%, 98%, or 99% identical to any of these particular dextransucrases). Such enzymatically produced dextran is linear (i.e., 100% alpha-1,6-linkages) and aqueous soluble.

The poly-1,6-glucan with branching can be produced enzymatically according to the procedures in WO 2015/183714 and WO 2017/091533 where, for example, alpha-1,2-branching enzymes such as “GTFJ18T1” or “GTF9905” can be added during or after the production of the dextran polymer (polysaccharide). It may be that any other enzyme known to produce alpha-1,2-branching can be added. For example, poly-1,6-glucan with alpha-1,3-branching can be prepared as disclosed in Vuillemin et al. (2016, J. Biol Chem. 291:7687-7702) or U.S. Appl. No. 62/871,796, which are incorporated herein by reference. The degree of branching of poly alpha-1,6-glucan or its derivative has less than or equal to 50%, 40%, 30%, 20%, 10%, or 5% (or any value between 5% and 50%) of short branching, for example alpha-1,2-branching, 1,3-branching, or both alpha-1,2-branching and alpha-1,3-branching. The degree of branching in a poly alpha-1,6-glucan starting material is maintained in a branched poly alpha-1,6-glucan ether formed by etherification of the branched poly alpha-1,6-glucan. The amount of alpha-1,2-branching or alpha-1,3-branching can be determined by NMR methods, as disclosed in the Test Methods below.

Without wishing to be bound by theory, it is believed that branching can increase the solubility of the poly alpha-1,6-glucan ether compound, which can lead to more convenient processability and/or transport. It is also believed that limits on the degree of branching can lead to improved performance in the final personal care composition.

A poly alpha-1,6-glucan ether compound may have a degree of alpha-1,2-branching that is less than 50%. A poly alpha-1,6-glucan ether compound may have a degree of alpha-1,2-branching that is at least 5%. From about 5% to about 50% of the backbone glucose monomer units of a poly alpha-1,6-glucan ether compound may have branches via alpha-1,2 or alpha-1,3 glycosidic linkages. From about 5% to about 35% of the backbone glucose monomer units of a poly alpha-1,6-glucan ether compound may have branches via alpha-1,2 or alpha-1,3 glycosidic linkages.

At least about 3%, may be at least about 5% of the backbone glucose monomer units of a poly alpha-1,6-glucan ether compound may have branches via alpha-1,2- or alpha-1,3-glycosidic linkages. A poly alpha-1,6-glucan ether compound may comprise a backbone of glucose monomer units wherein greater than or equal to 65% of the glucose monomer units are linked via alpha-1,6-glycosidic linkages. A poly alpha-1,6-glucan ether compound may comprise a backbone of glucose monomer units wherein greater than or equal to 65% of the glucose monomer units are linked via alpha-1,6-glycosidic linkages and at least 3%, may be at least 5%, may be from about 5% to about 30%, may be from about 5% to about 25%, may be from about 5% to about 20%, of the glucose monomer units have branches via alpha-1,2- or alpha-1,3-glycosidic linkages. A poly alpha-1,6-glucan ether compound may comprise a backbone of glucose monomer units wherein greater than or equal to 65% of the glucose monomer units are linked via alpha-1,6-glycosidic linkages and at least 5% of the glucose monomer units have branches via alpha-1,2 linkages. A poly alpha-1,6-glucan ether compound may comprise a backbone of glucose monomer units wherein greater than or equal to 65% of the glucose monomer units are linked via alpha-1,6-glycosidic linkages and at least 5% of the glucose monomer units have branches via alpha-1,3 linkages. A poly alpha-1,6-glucan ether compound may comprise a backbone of glucose monomer units wherein greater than or equal to 65% of the glucose monomer units are linked via alpha-1,6-glycosidic linkages and from about 5% to about 50% of the glucose monomer units have branches via alpha-1,2- or alpha-1,3-glycosidic linkages. A poly alpha-1,6-glucan ether compound may comprise a backbone of glucose monomer units wherein greater than or equal to 70% of the glucose monomer units are linked via alpha-1,6-glycosidic linkages and from about 5% to about 35% of the glucose monomer units have branches via alpha-1,2- or alpha-1,3-glycosidic linkages.

A poly alpha-1,6-glucan ether compound may comprise a backbone of glucose monomer units wherein greater than or equal to 90% of the glucose monomer units are linked via alpha-1,6-glycosidic linkages. A poly alpha-1,6-glucan ether compound may comprise a backbone of glucose monomer units wherein greater than or equal to 90% of the glucose monomer units are linked via alpha-1,6-glycosidic linkages and at least 5% of the glucose monomer units have branches via alpha-1,2- or alpha-1,3-glycosidic linkages. A poly alpha-1,6-glucan ether compound may comprise a backbone of glucose monomer units wherein greater than or equal to 90% of the glucose monomer units are linked via alpha-1,6-glycosidic linkages and at least 5% of the glucose monomer units have branches via alpha-1,2 linkages. A poly alpha-1,6-glucan ether compound may comprise a backbone of glucose monomer units wherein greater than or equal to 90% of the glucose monomer units are linked via alpha-1,6-glycosidic linkages and at least 5% of the glucose monomer units have branches via alpha-1,3 linkages. A poly alpha-1,6-glucan ether compound may comprise a backbone of glucose monomer units wherein greater than or equal to 90% of the glucose monomer units are linked via alpha-1,6-glycosidic linkages and from about 5% to about 50% of the glucose monomer units have branches via alpha-1,2- or alpha-1,3-glycosidic linkages. A poly alpha-1,6-glucan ether compound may comprise a backbone of glucose monomer units wherein greater than or equal to 90% of the glucose monomer units are linked via alpha-1,6-glycosidic linkages and from about 5% to about 35% of the glucose monomer units have branches via alpha-1,2- or alpha-1,3-glycosidic linkages.

The poly alpha-1,6-glucan and poly alpha-1,6-glucan ether compounds disclosed herein can have a number average degree of polymerization (DPn) in the range of 5 to 6000. The DPn can be in the range of from 5 to 100, or from 5 to 500, or from 5 to 1000, or from 5 to 1500, or from 5 to 2000, or from 5 to 2500, or from 5 to 3000, or from 5 to 4000, or from 5 to 5000, or from 5 to 6000. The DPn can be in the range of from 50 to 500, or from 50 to 1000, or from 50 to 1500, or from 50 to 2000, or from 50 to 3000, or from 50 to 4000, or from 50 to 5000, or from 50 to 6000.

The poly alpha-1,6-glucan and poly alpha-1,6-glucan ether compounds disclosed herein can have a weight average degree of polymerization (DPw) in the range of at least 5. The DPw can be in the range of from 5 to 6000, or from 50 to 5000, or from 100 to 4000, or from 250 to 3000, or from 500 to 2000, or from 750 to 1500, or from 1000 to 1400, or from 1100 to 1300. The DPw can be in the range of from 400 to 6000, or from 400 to 5000, or from 400 to 4000, or from 400 to 3000, or from 400 to 2000, or from 400 to 1500.

The poly alpha-1,6-glucan ether compounds disclosed herein can have a weight average molecular weight of from about 1000 to about 500,000 daltons, or from about 50,000 to about 500,000 daltons, or from about 10,000 to about 400,000 daltons, or from about 40,000 to about 300,000 daltons, or from about 80,000 to about 300,000 daltons, or from about 100,000 to about 250,000 daltons, or from about 150,000 to about 250,000 daltons, or from about 180,000 to about 225,000 daltons, or from about 180,000 to about 200,000 daltons. It may be that differently sized polymers may be used for different applications and/or intended benefits.

The poly alpha-1,6-glucan ether compounds disclosed herein can be derived from a poly alpha-1,6-glucan having a weight average molecular weight of from about 900 to about 450,000 daltons, determined prior to substitution with the least one positively charged organic group. The poly alpha-1,6-glucan ether compounds disclosed herein can be derived from a poly alpha-1,6-glucan having a weight average molecular weight of from about 5000 to about 400,000 daltons, or from about 10,000 to about 350,000 daltons, or from about 50,000 to about 350,000 daltons, or from about 90,000 to about 300,000 daltons, or from about 125,000 to about 250,000 daltons, or from about 150,000 to about 200,000 daltons. Differently sized feedstock or backbone polymers may be used for different applications, or depending on the intended degree of substitution.

The term “degree of substitution” (DoS) as used herein refers to the average number of hydroxyl groups substituted in each monomeric unit (glucose) of a cationic poly alpha-1,6-glucan ether compound, which includes the monomeric units within the backbone and within any alpha-1,2 or alpha-1,3 branches which may be present. Since there are at most three hydroxyl groups in a glucose monomeric unit in a poly alpha-1,6-glucan polymer or cationic poly alpha-1,6-glucan ether compound, the overall degree of substitution can be no higher than 3. It would be understood by those skilled in the art that, since a cationic poly alpha-1,6-glucan ether compound as disclosed herein can have a degree of substitution between about 0.001 to about 3.0, the substituents on the polysaccharide cannot only be hydroxyl. The degree of substitution of a poly alpha-1,6-glucan ether compound can be stated with reference to a specific substituent or with reference to the overall degree of substitution, that is, the sum of the DoS of each different substituent for an ether compound as defined herein. As used herein, when the degree of substitution is not stated with reference to a specific substituent or substituent type, the overall degree of substitution of the cationic poly alpha-1,6-glucan ether compound is meant. The degree of substitution may be a cationic degree of substitution, or even a net cationic degree of substitution. The target DoS can be chosen to provide the desired solubility and performance of a composition comprising a cationic poly alpha-1,6-glucan ether compound in the specific application of interest.

Cationic poly alpha-1,6-glucan ether compounds disclosed herein may have a DoS with respect to a positively charged organic group in the range of about 0.001 to about 3. A cationic poly alpha-1,6-glucan ether may have a DoS of about 0.01 to about 1.5. The poly alpha-1,6-glucan ether may have a DoS of about 0.01 to about 0.7. The poly alpha-1,6-glucan ether may have a DoS of about 0.01 to about 0.4. The poly alpha-1,6-glucan ether may have a DoS of about 0.01 to about 0.2. The DoS of the poly alpha-1,6-glucan ether compound can be at least about 0.001, 0.005, 0.1, 0.15, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0. The DoS may from about 0.01 to about 1.5, may be from about 0.01 to about 1.0, may be from about 0.01 to about 0.8, may be from about 0.03 to about 0.7, or from about 0.04 to about 0.6, or from about 0.05 to about 0.5. For performance reasons in personal care compositions, it may that the DoS be from about 0.01 to about 0.5, from about 0.01 to about 0.3, or from about 0.01 to about 0.25, or from about 0.01 to about 0.2, or from about 0.03 to about 0.15, or from about 0.04 to about 0.12. For performance reasons in the personal care composition, it may be for the DoS to be from about 0.01 to about 1, or from about 0.03 to about 0.8, or from about 0.04 to about 0.7, or from about 0.05 to about 0.6, or from about 0.2 to about 0.8, or from about 0.2 to about 0.6, or from about 0.3 to about 0.6, or from about 0.4 to about 0.6. The DoS of the poly alpha-1,6-glucan may be from 0.01 to about 0.6, may be from 0.02 to about 0.5.

Non-limiting examples of a cationically modified poly alpha-1,6-glucan ether compound may be cationically modified poly alpha-1,6-glucan ether compound having a DoS of 0.03, a charge density of 0.2 and a molecular weight of 185,000 daltons; may have a DoS of 0.06, a charge density of 0.35, and a molecular weight of 185,000 daltons; may have a a DoS of 0.07, a charge density of 0.4, and a molecular weight of 185,000 daltons; may have a DoS of 0.11, a charge density of 0.6, and a molecular weight of 185,000 daltons; may have a DoS of 0.14, a charge density of 0.75, and a molecular weight of 185,000 daltons; may have a DoS of 0.15, a charge density of 0.8, and a molecular weight of 185,000 daltons.

The cationic poly alpha-1,6-glucan ether compounds of the present disclosure may be characterized by a cationic charge density. Cationic charge density may be expressed as milliequivalents of charge per gram of compound (meq/mol) and may be determined according to the method provided in the Test Methods section. The cationic poly alpha-1,6-glucan ether compounds of the present disclosure may be characterized by a cationic charge density (or “CCD”) of from about 0.05 to about 12 meq/g, or from about 0.1 to about 8 meq/g, or from about 0.1 to about 4 meq/g, or from about 0.1 to about 3 meq/g, or from about 0.1 to about 2.6 meq/g.

A positively charged organic group comprises a chain of one or more carbons having one or more hydrogens substituted with another atom or functional group, wherein one or more of the substitutions is with a positively charged group. The term “chain” as used herein encompasses linear, branched, and cyclic arrangements of carbon atoms, as well as combinations thereof.

The poly alpha-1,6-glucan derivative comprises poly alpha-1,6-glucan substituted with at least one positively charged organic group on the polysaccharide backbone and/or on one or more of the optional branches. When substitution occurs on a glucose monomer contained in the backbone, the polysaccharide is derivatized at the 2, 3, and/or 4 glucose carbon position(s) with an organic group as defined herein which is linked to the polysaccharide through an ether (—O—) linkage in place of the hydroxyl group originally present in the underivatized (unsubstituted) poly alpha-1,6-glucan. When substitution occurs on a glucose monomer contained in a branch, the polysaccharide is derivatized at the 1, 2, 3, 4, or 6 glucose carbon position(s) with a positively charged organic group as defined herein which is linked to the polysaccharide through an ether (—O—) linkage.

A poly alpha-1,6-glucan ether compound as disclosed herein is termed a glucan “ether” herein by virtue of comprising the substructure —CG-O—CR—, wherein “—CG-” represents a carbon of a glucose monomer unit of a poly alpha-1,6-glucan ether compound, and wherein “—CR—” is comprised in the positively charged organic group. A cationic poly alpha-1,6-glucan monoether contains one type of a positively charged organic group. A cationic poly alpha-1,6-glucan mixed ether contains two or more types of positively charged organic groups. Mixtures of cationic poly alpha-1,6-glucan ether compounds can also be used.

Personal care compositions disclosed herein can comprise, or consist essentially of, one or more cationic poly alpha-1,6-glucan ether compounds as disclosed herein. A personal care composition may comprise one poly alpha-1,6-glucan ether compound. A personal care composition may comprise two or more poly alpha-1,6-glucan ether compounds, for example wherein the positively charged organic groups are different.

A personal care composition may comprise one or more cationic poly alpha-1,6-glucan ether compounds as disclosed herein, and may further comprise unsubstituted and/or non-cationic poly alpha-1,6-glucan compounds, which may be residual reactants that are unreacted/unsubstituted, or may have hydrolyzed. Typically, a low level of unsubstituted/non-cationic poly alpha-1,6-glucan compounds may be used, as low levels may be indicative of reaction completeness with regard to the substitution, and/or chemical stability of the compounds in the treatment composition. The weight ratio of the cationic poly alpha-1,6-glucan ether compounds to unsubstituted/non-cationic poly alpha 1,6-glucan compounds may be 95:5 or greater, may be 98:2 or greater, may be 99:1 or greater.

A “positively charged organic group” as used herein refers to a chain of one or more carbons that has one or more hydrogens substituted with another atom or functional group, wherein one or more of the substitutions is with a positively charged group. A positively charged group is typically bonded to the terminal carbon atom of the carbon chain. A positively charged organic group is considered to have a net positive charge since it comprises one or more positively charged groups, and comprises a cation (a positively charged ion). An organic group or compound that is “positively charged” typically has more protons than electrons and is repelled from other positively charged substances, but attracted to negatively charged substances. An example of a positively charged groups includes a substituted ammonium group. A positively charged organic group may have a further substitution, for example with one or more hydroxyl groups, oxygen atoms (forming a ketone group), alkyl groups, and/or at least one additional positively charged group.

A positively charged organic group may comprise a substituted ammonium group, which can be represented by Structure II:

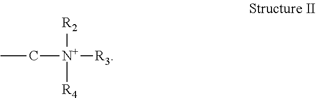

In Structure II, R₂, R₃ and R₄ may each independently represent a hydrogen atom, an alkyl group, or a C₆-C₂₄ aryl group. The carbon atom (C) shown in Structure II is part of the carbon chain of the positively charged organic group. The carbon atom is either directly ether-linked to a glucose monomer of poly alpha-1,6-glucan, or is part of a chain of two or more carbon atoms ether-linked to a glucose monomer of poly alpha-1,6-glucan. The carbon atom shown in Structure II can be —CH₂—, —CH— (where a H is substituted with another group such as a hydroxy group), or —C— (where both H's are substituted).

When R₂, R₃ and/or R₄ represent an alkyl group, the alkyl group can be a C₁-C₃₀ alkyl group, for example a methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, icosyl, henicosyl, docosyl, tricosyl, tetracosyl, C₂₅, C₂₆, C₂₇, C₂₈, C₂₉, or C₃₀ group. The alkyl group can be a C₁-C₂₄ alkyl group, or a C₁-C₁₈ or a C₆-C₂₀ alkyl group, or a C₁₀-C₁₆ alkyl group, or a C₁-C₄ alkyl group. When a positively charged organic group comprises a substituted ammonium group which has two or more alkyl groups, each alkyl group can be the same as or different from the other.

When R₂, R₃ and/or R₄ represent an aryl group, the aryl group can be a C₆-C₂₄ aryl group, optionally substituted with alkyl substituents. The aryl group can be a C₁₂-C₂₄ aryl group, optionally substituted with alkyl substituents, or a C₆-C₁₈ aryl group, optionally substituted with alkyl substituents.

A substituted ammonium group can be a “primary ammonium group”, “secondary ammonium group”, “tertiary ammonium group”, or “quaternary ammonium” group, depending on the composition of R₂, R₃ and R₄ in Structure II. A primary ammonium group is an ammonium group represented by Structure II in which each of R₂, R₃ and R₄ is a hydrogen atom (i.e., —C—NH3+).

A secondary ammonium group is an ammonium group represented by Structure II in which each of R₂ and R₃ is a hydrogen atom and R₄ is a C₁-C₃₀ alkyl group or a C₆-C₂₄ aryl group. A “secondary ammonium poly alpha-1,6-glucan ether compound” comprises a positively charged organic group having a monoalkylammonium group. A secondary ammonium poly alpha-1,6-glucan ether compound can be represented in shorthand as a monoalkylammonium poly alpha-1,6-glucan ether, for example monomethyl-, monoethyl-, monopropyl-, monobutyl-, monopentyl-, monohexyl-, monoheptyl-, monooctyl-, monononyl-, monodecyl-, monoundecyl-, monododecyl-, monotridecyl-, monotetradecyl-, monopentadecyl-, monohexadecyl-, monoheptadecyl-, or monooctadecyl-ammonium poly alpha-1,6-glucan ether. These poly alpha-1,6-glucan ether compounds can also be referred to as methyl-, ethyl-, propyl-, butyl-, pentyl-, hexyl-, heptyl-, octyl-, nonyl-, decyl-, undecyl-, dodecyl-, tridecyl-, tetradecyl-, pentadecyl-, hexadecyl-, heptadecyl-, or octadecyl-ammonium poly alpha-1,6-glucan ether compounds, respectively. An octadecyl ammonium group is an example of a monoalkylammonium group wherein each of R₂ and R₃ is a hydrogen atom and R₄ is an octadecyl group. It would be understood that a second member (i.e., R₁) implied by “secondary” in the above nomenclature is the chain of one or more carbons of the positively charged organic group that is ether-linked to a glucose monomer of poly alpha-1,6-glucan.

A tertiary ammonium group is an ammonium group represented by Structure II in which R₂ is a hydrogen atom and each of R₃ and R₄ is independently a C₁-C₂₄ alkyl group or a C₆-C₂₄ aryl group. The alkyl groups can be the same or different. A “tertiary ammonium poly alpha-1,6-glucan ether compound” comprises a positively charged organic group having a dialkylammonium group. A tertiary ammonium poly alpha-1,6-glucan ether compound can be represented in shorthand as a dialkylammonium poly alpha-1,6-glucan ether, for example dimethyl-, diethyl-, dipropyl-, dibutyl-, dipentyl-, dihexyl-, diheptyl-, dioctyl-, dinonyl-, didecyl-, diundecyl-, didodecyl-, ditridecyl-, ditetradecyl-, dipentadecyl-, dihexadecyl-, diheptadecyl-, or dioctadecyl-ammonium poly alpha-1,6-glucan ether. A didodecyl ammonium group is an example of a dialkyl ammonium group, wherein R₂ is a hydrogen atom and each of R₃ and R₄ is a dodecyl group. It would be understood that a third member (i.e., R₁) implied by “tertiary” in the above nomenclature is the chain of one or more carbons of the positively charged organic group that is ether-linked to a glucose monomer of poly alpha-1,6-glucan.

A quaternary ammonium group is an ammonium group represented by Structure II in which each of R₂, R₃ and R₄ is independently a C₁-C₃₀ alkyl group or a C₆-C₂₄ aryl group (i.e., none of R₂, R₃ and R₄ is a hydrogen atom).

A quaternary ammonium poly alpha-1,6-glucan ether compound may comprise a trialkyl ammonium group, where each of R₂, R₃ and R₄ is independently a C₁-C₃₀ alkyl group. The alkyl groups can all be the same, or two of the alkyl groups can be the same and one different from the others, or all three alkyl groups can be different from one another. A quaternary ammonium poly alpha-1,6-glucan ether compound can be represented in shorthand as a trialkylammonium poly alpha-1,6-glucan ether, for example trimethyl-, triethyl-, tripropyl-, tributyl-, tripentyl-, trihexyl-, triheptyl-, trioctyl-, trinonyl-, tridecyl-, triundecyl-, tridodecyl-, tritridecyl-, tritetradecyl-, tripentadecyl-, trihexadecyl-, triheptadecyl-, or trioctadecyl-ammonium poly alpha-1,6-glucan ether. It would be understood that a fourth member (i.e., R₁) implied by “quaternary” in this nomenclature is the chain of one or more carbons of the positively charged organic group that is ether-linked to a glucose monomer of poly alpha-1,6-glucan. A trimethylammonium group is an example of a trialkyl ammonium group, wherein each of R₂, R₃ and R₄ is a methyl group.

A positively charged organic group comprising a substituted ammonium group represented by Structure II can have each of R₂, R₃ and R₄ independently represent a hydrogen atom or an aryl group, such as a phenyl or naphthyl group, or an aralkyl group such as a benzyl group, or a cycloalkyl group such as cyclohexyl or cyclopentyl. Each of R₂, R₃ and R₄ may further comprise an amino group or a hydroxyl group.

The substituted ammonium group of the positively charged organic group is a substituent on a chain of one or more carbons that is ether-linked to a glucose monomer of the alpha-1,6-glucan. The carbon chain may contain from one to 30 carbon atoms. The carbon chain may be linear. Examples of linear carbon chains include, for example, —CH₂—, —CH₂CH₂—, —CH₂CH₂CH₂—, —CH₂(CH₂)₂CH₂—, —CH₂(CH₂)₃CH₂—, —CH₂(CH₂)₄CH₂—, —CH₂(CH₂)₅CH₂—, —CH₂(CH₂)₆CH₂—, —CH₂(CH₂)₇CH₂—, —CH₂(CH₂)₈CH₂—, —CH₂(CH₂)₉CH₂—, and —CH₂(CH₂)₁₀CH₂—; longer carbon chains can also be used, if desired. The carbon chain may be branched, meaning the carbon chain is substituted with one or more alkyl groups, for example methyl, ethyl, propyl, or butyl groups. The point of substitution can be anywhere along the carbon chain. Examples of branched carbon chains include —CH(CH₃)CH₂—, —CH(CH₃)CH₂CH₂—, —CH₂CH(CH₃)CH₂—, —CH(CH₂CH₃)CH₂—, —CH(CH₂CH₃)CH₂CH₂—, —CH₂CH(CH₂CH₃)CH₂—, —CH(CH₂CH₂CH₃)CH₂—, —CH(CH₂CH₂CH₃)CH₂CH₂—, and —CH₂CH(CH₂CH₂CH₃)CH₂—; longer branched carbon chains can also be used, if desired. Where the positively charged group is a substituted ammonium group, the first carbon atom in the chain is ether-linked to a glucose monomer of the poly alpha-1,6-glucan, and the last carbon atom of the chain in each of these examples is represented by the C in Structure II.

The chain of one or more carbons may be further substituted with one or more hydroxyl groups. Examples of a carbon chain having one or more substitutions with a hydroxyl group include hydroxyalkyl (e.g., hydroxyethyl, hydroxypropyl, hydroxybutyl, hydroxypentyl, hydroxyhexyl, hydroxyheptyl, hydroxyoctyl) groups and dihydroxyalkyl (e.g., dihydroxyethyl, dihydroxypropyl, dihydroxybutyl, dihydroxypentyl, dihydroxyhexyl, dihydroxyheptyl, dihydroxyoctyl) groups. Examples of hydroxyalkyl and dihydroxyalkyl (diol) carbon chains include —CH(OH)—, —CH(OH)CH₂—, —C(OH)₂CH₂—, —CH₂CH(OH)CH₂—, —CH(OH)CH₂CH₂—, —CH(OH)CH(OH)CH₂—, —CH₂CH₂CH(OH)CH₂—, —CH₂CH(OH)CH₂CH₂—, —CH(OH)CH₂CH₂CH₂—, —CH₂CH(OH)CH(OH)CH₂—, —CH(OH)CH(OH)CH₂CH₂— and —CH(OH)CH₂CH(OH)CH₂—. In each of these examples, the first carbon atom of the chain is ether-linked to a glucose monomer of poly alpha-1,6-glucan, and the last carbon atom of the chain is linked to a positively charged group. Where the positively charged group is a substituted ammonium group, the last carbon atom of the chain in each of these examples is represented by the C in Structure II.

An example of a quaternary ammonium poly alpha-1,6-glucan ether compound is trimethylammonium hydroxypropyl poly alpha-1,6-glucan. The positively charged organic group of this ether compound can be represented by the following structure:

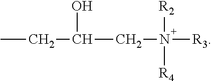

where each of R₂, R₃ and R₄ is a methyl group. The structure above is an example of a quaternary ammonium hydroxypropyl group.

Where a carbon chain of a positively charged organic group has a substitution in addition to a substitution with a positively charged group, such additional substitution may be with one or more hydroxyl groups, oxygen atoms (thereby forming an aldehyde or ketone group), alkyl groups (e.g., methyl, ethyl, propyl, butyl), and/or additional positively charged groups. A positively charged group is typically bonded to the terminal carbon atom of the carbon chain. A positively charged group can also comprise one or more imidazoline rings.

A cationic poly alpha-1,6-glucan ether compound as disclosed herein may be a salt. The counter ion for the positively charged organic group can be any suitable anion, including an acetate, borate, bromate, bromide, carbonate, chlorate, chloride, chlorite, dihydrogen phosphate, fluoride, hydrogen carbonate, hydrogen phosphate, hydrogen sulfate, hydrogen sulfide, hydrogen sulfite, hydroxide, hypochlorite, iodate, iodide, nitrate, nitride, nitrite, oxalate, oxide, perchlorate, permanganate, phosphate, phosphide, phosphite, silicate, stannate, stannite, sulfate, sulfide, sulfite, tartrate, or thiocyanate anion, may be chloride. In an aqueous solution, a poly alpha-1,6-glucan ether compound is in a cationic form. The positively charged organic groups of a cationic poly alpha-1,6-glucan ether compound can interact with salt anions that may be present in an aqueous solution.

The poly alpha-1,6-glucan ether compound may comprise a positively charged organic group, wherein the positively charged organic group comprises a substituted ammonium group. From about 0.5% to about 50% of the backbone glucose monomer units of the ether compound may have branches via alpha-1,2 glycosidic linkages, and the positively charged organic group may comprise a substituted ammonium group. From about 5% to about 30% of the backbone glucose monomer units of the ether compound may have branches via alpha-1,2 glycosidic linkages, and the substituted ammonium group may comprise a substituted ammonium group. From about 0.5% to about 50% of the backbone glucose monomer units of the ether compound may have branches via alpha-1,2 glycosidic linkages, and the substituted ammonium group may comprise a trimethyl ammonium group. From about 5% to about 35% of the backbone glucose monomer units of the ether compound may have branches via alpha-1,2 glycosidic linkages, and the substituted ammonium group may comprise a trimethyl ammonium group.

The poly alpha-1,6-glucan ether compound may comprise a positively charged organic group, wherein the positively charged organic group comprises a trimethylammonium hydroxyalkyl group. From about 0.5% to about 50% of the backbone glucose monomer units of the ether compound may have branches via alpha-1,2 glycosidic linkages, and the positively charged organic group may comprise a trimethylammonium hydroxyalkyl group. From about 5% to about 30% of the backbone glucose monomer units of the ether compound may have branches via alpha-1,2 glycosidic linkages, and the positively charged organic group may comprise a trimethylammonium hydroxyalkyl group. From about 0.5% to about 50% of the backbone glucose monomer units of the ether compound may have branches via alpha-1,2 glycosidic linkages, and the trimethylammonium hydroxyalkyl group may comprise a trimethylammonium hydroxypropyl group. From about 5% to about 30% of the backbone glucose monomer units of the ether compound may have branches via alpha-1,2 glycosidic linkages, and the trimethylammonium hydroxyalkyl group may comprise a trimethylammonium hydroxypropyl group.

The poly alpha-1,6-glucan ether compound may comprise a positively charged organic group, wherein the positively charged organic group comprises a substituted ammonium group comprising a quaternary ammonium group. From about 0.5% to about 50% of the backbone glucose monomer units of the ether compound may have branches via alpha-1,2 glycosidic linkages, and the quaternary ammonium group may comprise at least one C1 to C18 alkyl group. From about 5% to about 30% of the backbone glucose monomer units of the ether compound may have branches via alpha-1,2 glycosidic linkages, the quaternary ammonium group may comprise at least one C1 to C18 alkyl group. From about 0.5% to about 50% of the backbone glucose monomer units of the ether compound may have branches via alpha-1,2 glycosidic linkages, and the quaternary ammonium group may comprise at least one C1 to C₄ alkyl group. From about 5% to about 30% of the backbone glucose monomer units of the ether compound may have branches via alpha-1,2 glycosidic linkages, and the quaternary ammonium group may comprise at least one C1 to C₄ alkyl group. From about 0.5% to about 50% of the backbone glucose monomer units of the ether compound may have branches via alpha-1,2 glycosidic linkages, and the quaternary ammonium group may comprise at least one C10 to C16 alkyl group. From about 5% to about 30% of the backbone glucose monomer units of the ether compound may have branches via alpha-1,2 glycosidic linkages, and the quaternary ammonium group may comprise at least one C10 to C16 alkyl group.

The poly alpha-1,6-glucan ether compound may comprise a quaternary ammonium group comprising one C10 to C16 alkyl group, where the quaternary ammonium group further comprises two methyl groups. From about 0.5% to about 50% of the backbone glucose monomer units of the ether compound may have branches via alpha-1,2 glycosidic linkages, and the quaternary ammonium group may comprise one C10 to C16 alkyl group further comprises two methyl groups. From about 5% to about 30% of the backbone glucose monomer units of the ether compound may have branches via alpha-1,2 glycosidic linkages, and the quaternary ammonium group may comprise one C10 to C16 alkyl group further comprises two methyl groups.

From about 0.5% to about 50% of the backbone glucose monomer units of the ether compound may have branches via alpha-1,2 glycosidic linkages, and the quaternary ammonium group may comprise one C10 alkyl group and two methyl groups. From about 5% to about 30% of the backbone glucose monomer units of the ether compound may have branches via alpha-1,2 glycosidic linkages, and the quaternary ammonium group may comprise one C10 alkyl group and two methyl groups.

The poly alpha-1,6-glucan ether compound may comprise a positively charged organic group, wherein the positively charged organic group comprises a quaternary ammonium hydroxyalkyl group. From about 0.5% to about 50% of the backbone glucose monomer units of the ether compound may have branches via alpha-1,2 glycosidic linkages, and the positively charged organic group may comprise a quaternary ammonium hydroxyalkyl group. From about 5% to about 30% of the backbone glucose monomer units of the ether compound may have branches via alpha-1,2 glycosidic linkages, and the positively charged organic group may comprise a quaternary ammonium hydroxyalkyl group. From about 0.5% to about 50% of the backbone glucose monomer units of the ether compound may have branches via alpha-1,2 glycosidic linkages, and the quaternary ammonium hydroxyalkyl group may comprise a quaternary ammonium hydroxymethyl group, a quaternary ammonium hydroxyethyl group, or a quaternary ammonium hydroxypropyl group. From about 5% to about 30% of the backbone glucose monomer units of the ether compound may have branches via alpha-1,2 glycosidic linkages, and the quaternary ammonium hydroxyalkyl group may comprise a quaternary ammonium hydroxymethyl group, a quaternary ammonium hydroxyethyl group, or a quaternary ammonium hydroxypropyl group. From about 0.5% to about 50% of the backbone glucose monomer units of the ether compound may have branches via alpha-1,2 glycosidic linkages, and the quaternary ammonium hydroxyalkyl group may comprise a quaternary ammonium hydroxymethyl group. From about 5% to about 30% of the backbone glucose monomer units of the ether compound may have branches via alpha-1,2 glycosidic linkages, and the quaternary ammonium hydroxyalkyl group may comprise a quaternary ammonium hydroxymethyl group. From about 0.5% to about 50% of the backbone glucose monomer units of the ether compound may have branches via alpha-1,2 glycosidic linkages, and the quaternary ammonium hydroxyalkyl group may comprise a quaternary ammonium hydroxyethyl group. From about 5% to about 30% of the backbone glucose monomer units of the ether compound may have branches via alpha-1,2 glycosidic linkages, and the quaternary ammonium hydroxyalkyl group may comprise a quaternary ammonium hydroxyethyl group. From about 0.5% to about 50% of the backbone glucose monomer units of the ether compound may have branches via alpha-1,2 glycosidic linkages, and the quaternary ammonium hydroxyalkyl group may comprise a quaternary ammonium hydroxypropyl group. From about 5% to about 30% of the backbone glucose monomer units of the ether compound may have branches via alpha-1,2 glycosidic linkages, and the quaternary ammonium hydroxyalkyl group may comprise a quaternary ammonium hydroxypropyl group.

Poly alpha-1,6-glucan ether compounds containing a positively charged organic group, such as a trimethyl ammonium group, a substituted ammonium group, or a quaternary ammonium group, can be prepared using methods similar to those disclosed in published patent application US 2016/0311935, which is incorporated herein by reference in its entirety. US 2016/0311935 discloses poly alpha-1,3-glucan ether compounds comprising positively charged organic groups and having a degree of substitution of up to about 3.0, as well as methods of producing such ether compounds. Cationic poly alpha-1,6-glucan ethers may be prepared by contacting poly alpha-1,6-glucan with at least one etherification agent comprising a positively charged organic group under alkaline conditions. For example, alkaline conditions may be prepared by contacting the poly alpha-1,6-glucan with a solvent and one or more alkali hydroxides to provide a solution or mixture, and at least one etherification agent is then added. As another example, at least one etherification agent can be contacted with poly alpha-1,6-glucan and solvent, and then the alkali hydroxide can be added. The mixture of poly alpha-1,6-glucan, etherification agent, and alkali hydroxides can be maintained at ambient temperature or optionally heated, for example to a temperature between about 25° C. and about 200° C., depending on the etherification agent and/or solvent employed. Reaction time for producing a poly alpha-1,6-glucan ether will vary corresponding to the reaction temperature, with longer reaction time necessary at lower temperatures and lower reaction time necessary at higher temperatures.

Typically, the solvent comprises water. Optionally, additional solvent can be added to the alkaline solution, for example alcohols such as isopropanol, acetone, dioxane, and toluene. Alternatively, solvents such as lithium chloride (LiCl)/N,N-dimethyl-acetamide (DMAc), SO₂/diethylamine (DEA)/dimethyl sulfoxide (DMSO), LiCl/1,3-dimethy-2-imidazolidinone (DMI), N,N-dimethylformamide (DMF)/N₂O₄, DMSO/tetrabutyl-ammonium fluoride trihydrate (TBAF), N-methylmorpholine-N-oxide (NMMO), Ni(tren)(OH)2 [tren-tris(2-aminoethyl)amine]aqueous solutions and melts of LiClO₄·3H₂O, NaOH/urea aqueous solutions, aqueous sodium hydroxide, aqueous potassium hydroxide, formic acid, and ionic liquids can be used.

An etherification agent may be one that can etherify poly alpha-1,6-glucan with a positively charged organic group, where the carbon chain of the positively charged organic group only has a substitution with a positively charged group (e.g., substituted ammonium group such as trimethylammonium). Examples of such etherification agents include dialkyl sulfates, dialkyl carbonates, alkyl halides (e.g., alkyl chloride), iodoalkanes, alkyl triflates (alkyl trifluoromethanesulfonates) and alkyl fluorosulfonates, where the alkyl group(s) of each of these agents has one or more substitutions with a positively charged group (e.g., substituted ammonium group such as trimethylammonium). Other examples of such etherification agents include dimethyl sulfate, dimethyl carbonate, methyl chloride, iodomethane, methyl triflate and methyl fluorosulfonate, where the methyl group(s) of each of these agents has a substitution with a positively charged group (e.g., substituted ammonium group such as trimethylammonium). Other examples of such etherification agents include diethyl sulfate, diethyl carbonate, ethyl chloride, iodoethane, ethyl triflate and ethyl fluorosulfonate, where the ethyl group(s) of each of these agents has a substitution with a positively charged group (e.g., substituted ammonium group such as trimethylammonium). Other examples of such etherification agents include dipropyl sulfate, dipropyl carbonate, propyl chloride, iodopropane, propyl triflate and propyl fluorosulfonate, where the propyl group(s) of each of these agents has one or more substitutions with a positively charged group (e.g., substituted ammonium group such as trimethylammonium). Other examples of such etherification agents include dibutyl sulfate, dibutyl carbonate, butyl chloride, iodobutane and butyl triflate, where the butyl group(s) of each of these agents has one or more substitutions with a positively charged group (e.g., substituted ammonium group such as trimethylammonium). Other examples of etherification agents include halides of imidazoline-ring-containing compounds.

An etherification agent may be one that can etherify poly alpha-1,6-glucan with a positively charged organic group, where the carbon chain of the positively charged organic group has a substitution, for example a hydroxyl group, in addition to a substitution with a positively charged group, for example a substituted ammonium group such as trimethylammonium. Examples of such etherification agents include hydroxyalkyl halides (e.g., hydroxyalkyl chloride) such as hydroxypropyl halide and hydroxybutyl halide, where a terminal carbon of each of these agents has a substitution with a positively charged group (e.g., substituted ammonium group such as trimethylammonium); an example is 3-chloro-2-hydroxypropyl-trimethylammonium. Additional examples of etherification agents comprising a positively charged organic group include 2,3-epoxypropyltrimethylammonium chloride, 3-chloro-2-hydroxypropyl dodecyldimethylammonium chloride, 3-chloro-2-hydroxypropyl cocoalkyldimethylammonium chloride, 3-chloro-2-hydroxypropyl stearyldimethylammonium chloride, and quaternary ammonium compounds such as halides of imidazoline-ring-containing compounds. Other examples of such etherification agents include alkylene oxides such as propylene oxide (e.g., 1,2-propylene oxide) and butylene oxide (e.g., 1,2-butylene oxide; 2,3-butylene oxide), where a terminal carbon of each of these agents has a substitution with a positively charged group (e.g., substituted ammonium group such as trimethylammonium).

When producing a poly alpha-1,6-glucan ether compound comprising two or more different positively charged organic groups, two or more different etherification agents would be used, accordingly. Any of the etherification agents disclosed herein may be combined to produce poly alpha-1,6-glucan ether compounds having two or more different positively charged organic groups. Such two or more etherification agents may be used in the reaction at the same time, or may be used sequentially in the reaction. When used sequentially, any of the temperature-treatment (e.g., heating) steps may optionally be used between each addition. Sequential introduction of etherification agents may be used to control the desired DoS of each positively charged organic group. In general, a particular etherification agent would be used first if the organic group it forms in the ether product is desired at a higher DoS compared to the DoS of another organic group to be added.

The amount of etherification agent to be contacted with poly alpha-1,6-glucan in a reaction under alkaline conditions can be selected based on the degree of substitution desired in the ether compound. The amount of ether substitution groups on each monomeric unit in poly alpha-1,6-glucan ether compounds can be determined using nuclear magnetic resonance (NMR) spectroscopy. In general, an etherification agent can be used in a quantity of at least about 0.05 mole per mole of poly glucan. There may be no upper limit to the quantity of etherification agent that can be used.

Reactions for producing poly alpha-1,6-glucan ether compounds can optionally be carried out in a pressure vessel such as a Parr reactor, an autoclave, a shaker tube, or any other pressure vessel well known in the art. Optionally, poly alpha-1,6-glucan ether compounds can be prepared under an inert atmosphere, with or without heating. As used herein, the term “inert atmosphere” refers to a nonreactive gas atmosphere such as nitrogen, argon, or helium.

After contacting the poly alpha-1,6-glucan, solvent, alkali hydroxide, and etherification reagent for a sufficient reaction time to produce a poly alpha-1,6-glucan ether compound, the reaction mixture can optionally be filtered by any means known in the art which allows removal of liquids from solids.

Following etherification, one or more acids may be optionally added to the reaction mixture to lower the pH to a neutral pH range that is neither substantially acidic nor substantially acidic, for example a pH of about 6-8, or about 6.0, 6.2, 6.4, 6.6, 6.8, 7.0, 7.2, 7.4, 7.6, 7.8, or 8.0, if desired. Various acids useful for this purpose include sulfuric, acetic, hydrochloric, nitric, any mineral (inorganic) acid, any organic acid, or any combination of these acids.

A poly alpha-1,6-glucan ether compound can optionally be washed one or more times with a liquid that does not readily dissolve the compound. For example, a poly alpha-1,6-glucan ether can be washed with water, alcohol, isopropanol, acetone, aromatics, or any combination of these, depending on the solubility of the ether compound therein (where lack of solubility is desirable for washing). In general, a solvent comprising an organic solvent such as alcohol may be used for the washing. A poly alpha-1,6-glucan ether product can be washed one or more times with an aqueous solution containing methanol or ethanol, for example. For example, 70-95 wt % ethanol can be used to wash the product. A poly alpha-1,6-glucan ether product may be washed with a methanol:acetone (e.g., 60:40) solution.

A poly alpha-1,6-glucan ether compound can optionally purified by membrane filtration.

A poly alpha-1,6-glucan ether produced using the methods disclosed above can be isolated. This step can be performed before or after neutralization and/or washing steps using a funnel, centrifuge, press filter, or any other method or equipment known in the art that allows removal of liquids from solids. An isolated poly alpha-1,6-glucan ether product can be dried using any method known in the art, such as vacuum drying, air drying, or freeze drying.

Any of the above etherification reactions can be repeated using a poly alpha-1,6-glucan ether product as the starting material for further modification. This approach may be suitable for increasing the DoS of a positively charged organic group, and/or adding one or more different positively charged organic groups to the ether product. Also, this approach may be suitable for adding one or more organic groups that are not positively charged, such as an alkyl group (e.g., methyl, ethyl, propyl, butyl) and/or a hydroxyalkyl group (e.g., hydroxyethyl, hydroxypropyl, hydroxybutyl). Any of the above etherification agents, but without the substitution with a positively charged group, can be used for this purpose.

As described above, materials derived from sustainable/renewable feedstock materials are often desirable. Similarly, biodegradable materials may also be used. For example, biodegradable cationic poly alpha-1,6-glucan ether compounds may be used over non-biodegradable materials from an environmental footprint perspective. The biodegradability of a material can be evaluated by methods known in the art, for example as disclosed in the Biodegradability Test Method section herein below. The cationic poly alpha-1,6-glucan ether compound may be characterized by a biodegradability as determined by the Biodegradability Test Method below (i.e., Carbon Dioxide Evolution Test Method—OECD Guideline 301B) of at least 10% on the 90th day of the test duration. The cationic poly alpha-1,6-glucan ether compound may be characterized by a biodegradability, as determined by the Biodegradability Test Method below, of at least 5%, 10%, 15%, 20%, 25%, 30%, 35%, 40%, 45%, 50%, 55%, 60%, 65%, 70%, 75%, or 80%, or any value between 5% and 80%, on the 90th day of the test duration. The cationic poly alpha-1,6-glucan ether compound may be characterized by a biodegradability, as determined by the Biodegradability Test Method below, of at least 5%, 10%, 15%, 20%, 25%, 30%, 35%, 40%, 45%, 50%, 55%, or 60%, or any value between 5% and 60%, on the 60th day of the test duration. Without wishing to be bound by theory, it is believed that the biodegradability profile of the presently described materials may be affected by the degree of substitution, the molecular weight, the degree of branching, and/or the solubility of the material. For example, it is believed that relatively lower degrees of substitution (e.g., lower cationic charge density) and/or increased solubility will be associated with higher degrees of biodegradability.

### Carrier

The personal care composition may optionally include 20-95% of an aqueous carrier such as water and/or a water miscible solvent. The type and amount of aqueous carrier should be selected to provide the composition with the desired rheological properties. The liquid carrier can be water with, e.g., less than 10%, 7%, 5%, 3%, 1%, 0.5% or even 0% miscible organic solvent. Some nonlimiting examples of organic solvents include lower alkyl alcohols (e.g., ethanol and isopropanol) and polyhydric alcohols (e.g., propylene glycol, hexylene glycol, glycerin, and propane diol).

### Additional Ingredients

The personal care compositions described herein may include a variety of optional ingredients to tailor the properties and characteristics of the composition, as desired. The optional ingredients may be materials that are commonly included in compositions of the type. The optional ingredients should be physically and chemically compatible with the essential components of the personal care composition and should not otherwise unduly impair the stability, aesthetics, or performance of the composition. Individual concentrations of optional components can generally range from 0.001% to 10%.

Some non-limiting examples of optional ingredients that can be included in the personal care compositions herein include deposition aids, cationic polymers, conditioning agents (including gel network, triglyceride oils, hydrocarbon oils, fatty esters, silicones), anti-microbial agents, suspending agents, viscosity modifiers, dyes, pigments, nonvolatile solvents or diluents (water soluble and insoluble), pearlescent aids, foam boosters, pediculocides, pH adjusting agents, perfumes, preservatives, chelants, proteins, vitamins, amino acids, skin active agents, sunscreens, UV absorbers, stabilizers, and combinations of these.

### Test Methods

### Measurement of Scalp Care Active Deposition

Scalp care active deposition in-vivo on scalp can be determined by extraction, using either ethanol or EDTA, of the agent after the scalp has been treated with a surfactant-soluble agent containing cleansing composition and rinsed off. The concentration of agent in the ethanol or EDTA extraction solvent is measured by HPLC. Quantitation is made by reference to a standard curve. The concentration detected by HPLC is converted into an amount collected in grams by using the concentration multiplied by volume.
The percent agent deposited can be calculated using the following equation:

$$\% agent deposited = \frac{\frac{grams of agent deposited}{area of scalp extracted}}{\frac{( wt . \% agent in shampoo ) \times ( grams of shampoo applied )}{area of scalp treated}} \times 100 \%$$

Sample Calculation for % Piroctone Olamine deposited, where:

$$Grams of agent deposited = 1.7 \times {10}^{- 6} g$$

Area of scalp extracted=1 cm²
Wt % Piroctone Olamine in shampoo=1.0%
Grams of shampoo applied=5 g
Area of scalp treated=300 cm²

$$\begin{matrix}\% Piroctone Olamine deposited = \frac{\frac{1.7 \times {10}^{- 6} g}{1 {cm}^{2}}}{\frac{( 1. \% ) \times ( 5 g )}{300 {cm}^{2}}} \times 100 \% \\ \% Piroctone olamine deposited = 1.02 \%\end{matrix}$$

The deposition efficiency can be calculated using the following equation:

$$Deposition efficiency = \frac{\% agent deposited by example formula}{\% agent deposited by control formula}$$

Sample calculation for deposition efficiency, where:
% Piroctone Olamine deposited by example formula=1.92%
% Piroctone Olamine deposited by control formula=1.02%

$$\begin{matrix}Deposition efficiency = \frac{1.92 \%}{1.02 \%} \\ Deposition efficiency = 1.9 X\end{matrix}$$

### % Transmittance Method

Techniques for analysis of formation of complex coacervates are known in the art. One method to assess coacervate formation upon dilution for a transparent or translucent composition is to use a spectrophotometer to measure the percentage of light transmitted through the diluted sample (% T). As percent light transmittance (% T) values measured of the dilution decrease, typically higher levels of coacervate are formed. Dilutions samples at various weight ratios of water to composition can be prepared, for example 2 parts of water to 1 part composition (2:1), or 7.5 parts of water to 1 part composition (7.5:1), or 16 parts of water to 1 part composition (16:1), or 34 parts of water to 1 part composition (34:1), and the % T measured for each dilution ratio sample. Examples of possible dilution ratios may include 2:1, 3:1, 5:1, 7.5:1, 11:1, 16:1, 24:1, or 34:1. By averaging the % T values for samples that span a range of dilution ratios, it is possible to simulate and ascertain how much coacervate a composition on average would form as a consumer applies the composition to wet hair, lathers, and then rinses it out. Average % T can be calculated by taking the numerical average of individual % T measurements for the following dilution ratios: 2:1, 3:1, 5:1, 7.5:1, 11:1, 16:1, 24:1, and 34:1.

% T can be measured using Ultra-Violet/Visible (UV/VI) spectrophotometry which determines the transmission of UV/VIS light through a sample. A light wavelength of 600 nm has been shown to be adequate for characterizing the degree of light transmittance through a sample. Typically, it is best to follow the specific instructions relating to the specific spectrophotometer being used. In general, the procedure for measuring percent transmittance starts by setting the spectrophotometer to 600 nm. Then a calibration “blank” is run to calibrate the readout to 100 percent transmittance. A single test sample is then placed in a cuvette designed to fit the specific spectrophotometer and care is taken to ensure no air bubbles are within the sample before the % T is measured by the spectrophotometer at 600 nm. In the present invention, multiple samples are be measured simultaneously by using a spectrophotometer such as the SpectraMax M-5 available from Molecular Devices. Multiple dilution samples can be prepared within a 96 well plate (VWR catalog #82006-448) and then transferred to a 96 well visible flat bottom plate (Greiner part #655-001), ensuring that no air bubbles are within the sample. The flat bottom plate is placed within the SpectraMax M-5 and % T measured using the Software Pro v.5™ software available from Molecular Devices.

### Results

### Scalp Care Active Deposition

Two personal care compositions, a Control and Example 3 (defined in example table below), are tested to understand how piroctone olamine deposition is impacted by adding a cationically modified poly alpha-1,6-glucan ether compound (Alpha-Glucan Hydroxypropyltrimonium Chloride, supplier: IFF; (MW=185 kDa, CD=0.75, DoS=0.14)) to the formula. The samples are tested via an in-vivo scalp deposition test, with each code having a total of 60 observations. The deposition data is analyzed with a mixed model. Prior to analysis the data is transformed using natural log. A mixed model included factors for panelist and shampoo formulation, where panelist are treated as a random effect and shampoo formulation as a fixed effect. A t-test is used to assess the significance of the pairwise comparisons between shampoo formulations, confirming there is statistically significant more piroctone olamine deposition from Example 3 when compared to the Control. The results of this testing have demonstrated in the table below that adding 0.75% of cationically modified poly alpha-1,6-glucan ether compound of Example 3 (Alpha-Glucan Hydroxypropyltrimonium Chloride, supplier: IFF; (MW=185 kDa, CD=0.75, DoS=0.14)) to an otherwise identical composition, results in a 12.6% increase in piroctone olamine deposition on the scalp.

| | Guar | | | | | |
|---|---|---|---|---|---|---|
| | Hydroxypropyl | Alpha-Glucan | Piroctone | PO | | |
| | Trimonium | Hydroxypropyltrimonium | Olamine | Deposition | | |
| | chloride * | Chloride ** | (PO) | (ug/cm²) | Statistics | |
| Control | 0.50% | 0.00% | 0.50% | 2.31 | A | |
| Example 3 | 0.50% | 0.75% | 0.50% | 2.6 | | B |

* Jaguar ® Exel polymer from Syensqo
** Example 3 included Alpha-Glucan Hydroxypropyltrimonium Chloride, supplier: IFF; (MW = 185 kDa, CD = 0.75, DoS = 0.14)

In the present invention, a personal care composition may have at least a 10% increase in deposition when compared to a composition with to a composition with no cationically modified poly alpha-1,6-glucan ether compound; a personal care composition may have at least a 20% increase in deposition when compared to a composition with to a composition with no cationically modified poly alpha-1,6-glucan ether compound. In the present invention, a personal care composition may have at least a 1× (times) increase in deposition, or at least a 2× (times) increase in deposition, or a 3× (times) increase in deposition when compared to a composition with to a composition with no cationically modified poly alpha-1,6-glucan ether compound.

### % Transmittance and Coacervate

| | % T @ 1:1 | % T @ 1:2.5 | % T @ 1:3 |
|---|---|---|---|
| | dilution after | dilution after | dilution after |
| Sample | 5 min | 5 min | 5 min |
| Control | 65.9 | 27.5 | 20.8 |
| Example 7* | 30.5 | 19.3 | 18.9 |

*Example 7 includes Alpha-Glucan Hydroxypropyltrimonium Chloride, supplier: IFF; (MW = 185 kDa, CD = 0.75, DoS = 0.14)

The % T value serves as an indicator of the amount of coacervate formed. A lower % T indicates less light transmission, which corresponds to a higher amount of coacervate formation. The measurements are made with Example 7, defined in example table below, which is similar to Example 3 but with no glycol distearte, as this material is not desirable for measuring % T. The table above demonstrates that when the cationically modified poly alpha-1,6-glucan ether compound of Example 7 (Alpha-Glucan Hydroxypropyltrimonium Chloride, supplier: IFF; (MW=185 kDa, CD=0.75, DoS=0.14) is added alongside 0.5% Jaguar® Exel polymer in the formula at dilutions of 1:1, 1:1.5, and 1:2, the % T is lower, indicating increased coacervation compared to using only 0.5% Jaguar® Exel polymer. This increased coacervation resulting from the addition of cationically modified poly alpha-1,6-glucan ether compound of Example 7 (Alpha-Glucan Hydroxypropyltrimonium Chloride, supplier: IFF; (MW=185 kDa, CD=0.75, DoS=0.14) may facilitate the deposition of the scalp care active.

### Preparation of Personal Care Compositions

The personal care compositions are prepared by adding surfactants, scalp care actives, perfume, viscosity modifiers, cationic polymers and the remainder of the water with ample agitation to ensure a homogenous mixture. The mixture can be heated to 50-75° C. to speed the solubilization of the soluble agents, then cooled. Product pH may be adjusted as necessary to provide shampoo compositions of the present invention which are suitable for application to human hair and scalp, and may vary from about pH 4 to 9, or from about pH 4 to 6, or from about pH 4.0 to 5.5, based on the selection of particular detersive surfactants and/or other components.

### Product Form

The personal care compositions of the present invention may be presented in typical personal care formulations. They may be in the form of solutions, dispersion, emulsions, powders, talcs, encapsulated, spheres, spongers, solid dosage forms, foams, and other delivery mechanisms. The compositions of the present invention may be hair tonics, leave-on hair products such as treatment, and styling products, rinse-off hair products such as shampoos and personal cleansing products, and treatment products; and any other form that may be applied to hair.

### Non-Limiting Examples

The personal care compositions illustrated in the following examples are prepared by conventional formulation and mixing methods. All exemplified amounts are listed as weight percents on an active basis and exclude minor materials such as diluents, preservatives, color solutions, imagery ingredients, botanicals, and so forth, unless otherwise specified. All percentages are based on weight unless otherwise specified.

| | Example | Example | Example | Example |
|---|---|---|---|---|
| Ingredients | 1 | 2 | 3 | 4 |
| Alpha Olefin Sulfonate¹ | — | 9.00 | 5.00 | 5.00 |
| Lauramidopropyl Betaine² | — | | — | 1.00 |
| Sodium Lauryl Sulfate ³ | 9.00 | | 5.00 | 5.00 |
| Cocamidopropyl Betaine ⁴ | 3.00 | 5.00 | 2.00 | 1.00 |
| Cocamide MEA ⁵ | — | 1.00 | — | 1.00 |
| Piroctone Olamine⁶ | 0.80 | 0.50 | 0.50 | 0.50 |
| Zinc pyrithione (ZPT) ⁷ | | | | 0.50 |
| Guar Hydroxypropyltrimonium Chloride⁸ | 0.50 | | 0.5 | — |
| Polyquaternium 10 ⁹ | | 0.25 | | 0.25 |
| Alpha-Glucan Hydroxypropyltrimonium | 0.75 | 0.75 | 0.75 | 0.75 |
| Chloride ¹⁰ | | | | |
| Alpha-Glucan Hydroxypropyltrimonium | | | | |
| Chloride ¹¹ | | | | |
| Dimethicone ¹² | 0.50 | 0.80 | — | 0.50 |
| Glycol Distearate ¹³ | 1.50 | — | 2.5 | — |
| Trihydroxystearin ¹⁴ | — | 0.10 | — | 0.08 |
| Sodium Benzoate ¹⁵ | 0.25 | 0.15 | 0.15 | 0.15 |
| Methylchloroisothiazolinone/ | 0.005 | — | — | — |
| Methylisothiazolinone ¹⁶ | | | | |
| Sodium Hydroxide ¹⁷ | QS | QS | QS | QS |
| Citric Acid ¹⁸ | QS | QS | QS | QS |
| Hydrochloric acid ¹⁹ | QS | QS | QS | QS |
| Sodium Chloride ²⁰ | QS | QS | QS | QS |
| Sodium Xylene Sulfonate ²¹ | QS | QS | QS | QS |
| Salicylic Acid ²² | — | — | — | — |
| Tetrasodium EDTA²³ | 0.10 | 0.10 | 0.10 | 0.10 |
| Sodium Salicylate ²⁴ | — | 0.15 | 0.15 | 0.15 |
| Fragrance | 1.00 | 1.00 | 1.00 | 1.00 |
| Water | QS | QS | QS | QS |

| | Example | Example | Example |
|---|---|---|---|
| Ingredients | 5 | 6 | 7 |
| Alpha Olefin Sulfonate¹ | 4.00 | — | 5.00 |
| Lauramidopropyl Betaine² | 4.00 | — | — |
| Sodium Lauryl Sulfate ³ | 4.00 | 13.00 | 5.00 |
| Cocamidopropyl Betaine ⁴ | — | 1.00 | 2.00 |
| Cocamide MEA ⁵ | 2.00 | — | — |
| Piroctone Olamine⁶ | 0.80 | 0.50 | 0.50 |
| Zinc pyrithione (ZPT) ⁷ | | — | |
| Guar Hydroxypropyltrimonium Chloride⁸ | 0.40 | 0.50 | 0.5 |
| Polyquaternium 10 ⁹ | | | |
| Alpha-Glucan Hydroxypropyltrimonium Chloride ¹⁰ | 0.75 | | 0.75 |
| Alpha-Glucan Hydroxypropyltrimonium Chloride ¹¹ | | 0.75 | |
| Dimethicone ¹² | 0.80 | 0.50 | — |
| Glycol Distearate ¹³ | 1.50 | 1.00 | — |
| Trihydroxystearin ¹⁴ | — | 0.10 | — |
| Sodium Benzoate ¹⁵ | 0.25 | 0.15 | 0.15 |
| Methylchloroisothiazolinone/ | 0.005 | | — |
| Methylisothiazolinone ¹⁶ | | | |
| Sodium Hydroxide ¹⁷ | QS | QS | QS |
| Citric Acid ¹⁸ | QS | QS | QS |
| Hydrochloric acid ¹⁹ | QS | QS | QS |
| Sodium Chloride ²⁰ | QS | QS | QS |
| Sodium Xylene Sulfonate ²¹ | QS | QS | QS |
| Salicylic Acid ²² | 0.5 | | — |
| Tetrasodium EDTA²³ | 0.10 | 0.10 | 0.10 |
| Sodium Salicylate ²⁴ | — | | 0.15 |
| Fragrance | 1.00 | 1.00 | 1.00 |
| Water | QS | QS | QS |

| | |
|---|---|
| 1 | Bioterge AOS-40 at 40% active, supplier: Solvay |
| 2 | Mackam DAB-ULS at 35%, supplier: Solvay |
| 3 | Sodium Lauryl Sulfate at 29% active, supplier: P&G |
| 4 | Tego Betain L 7 OK at 30% active, supplier: Evonik |
| 5 | Ninol Comf at 85% active, supplier: Stepan |
| 6 | Octopirox, supplier: Clariant |
| 7 | Zinc Pyrithione (ZPT) at 40% active, supplier: Arch Chemicals |
| 8 | JAQUAR ® Excel Polymer, supplier: Syensqo |
| 9 | UCARE Polymer JR-30M, supplier: Dow |
| 10 | Alpha-Glucan Hydroxypropyltrimonium Chloride, supplier: IFF; (MW = 185 kDa, CD = |
| | 0.75, DoS = 0.14 |
| 11 | Alpha-Glucan Hydroxypropyltrimonium Chloride, supplier: IFF; (MW = 185 kDa, CD = |
| | 0.4, DoS = 0.07) |
| 12 | CF330M, supplier: Momentive |
| 13 | TEGIN G 1100, supplier: Evonik |
| 14 | Thixcin R, Supplier Elementis |
| 15 | Sodium Benzoate Dense NF/FCC, supplier: Emerald Performance Materials |
| 16 | Kathon CG at 1.5% active, supplier: Dow |
| 17 | Sodium Hydroxide - Caustic Soda at 50% active, supplier: K.A. Steel Chemicals, Inc.; |
| | level adjustable as process aid or to achieve target pH |
| 18 | Citric Acid Anhydrous, supplier: Archer Daniels Midland; level adjustable to achieve |
| | target pH |
| 19 | 6N HCl, supplier: J.T. Baker, level adjustable to achieve target pH |
| 20 | Sodium Chloride, supplier: Morton; level adjustable to achieve target viscosity |
| 21 | Stepanate SXS at 40%, supplier: Stepan |
| 22 | Salicylic Acid, supplier: Salicylates and Chemicals |
| 23 | Dissolvine 220-S at 84% active, Supplier: Akzo Nobel |
| 24 | Sodium Salicylate, supplier: JQC (Huayin) Pharmaceutical Co., Ltd. |

### Additional Examples/Combinations

A. A personal care composition comprising
   a. from about 8 to about 20% of one or more surfactants;
   b. from about 0.01 to about 10% of scalp care active;
   c. from about 0.01 to about 5% of one or more cationic polymer;
   d. from about 0.1% to about 5% of a cationically modified poly alpha-1,6-glucan ether compound; and wherein the composition has an increase in deposition of the scalp care active when compared to a control composition with no cationically modified poly alpha-1,6-glucan ether compound.
B. A personal care composition according to Paragraph A, wherein the cationically modified poly alpha-1,6-glucan ether compound:
   comprises a cationically modified poly alpha-1,6-glucan substituted with at least one positively charged organic group;
   comprises a backbone of glucose monomer units wherein at least 65% of the backbone glucose monomer units are linked via alpha-1,6-glycosidic linkages;
   has a degree of substitution of from 0.001 to 3; and
   is characterized by one or more of the following i-iii:
      i. a weight average degree of polymerization of from 5 to 6000,
      ii. a weight average molecular weight of from 1000 to 500,000 daltons,
      iii. derived from a poly alpha-1,6-glucan having a weight average molecular weight of from 900 to 450,000 daltons determined prior to substitution with the least one positively charged organic group.
C. A personal care composition according to Paragraph A-B, wherein from about 5% to about 35% of the backbone glucose monomer units have branches via alpha-1,2 and/or alpha-1,3-glycosidic linkages.
D. A personal care composition according to Paragraph A-C, wherein the positively charged organic group comprises a substituted ammonium group.
E. A personal care composition according to Paragraph A-D, wherein the substituted ammonium group comprises a quaternary ammonium group comprising at least one C10 to C16 alkyl group.
F. A personal care composition according to Paragraph A-E, wherein the substituted ammonium group comprises a quaternary ammonium group comprising a trimethylammonium group.
G. A personal care composition according to Paragraph A-F, wherein the positively charged organic group comprises a quaternary ammonium hydroxyalkyl group.
H. A personal care composition according to Paragraph A-G, comprising from about from 0.01% to 5% of the cationically modified poly alpha-1,6-glucan ether compound by weight of the personal care composition.
I. A personal care composition according to Paragraph A-H, wherein the cationically modified poly alpha-1,6-glucan ether compound has a degree of substitution of from about 0.01 to about 0.3.
J. A personal care composition according to Paragraph A-I, wherein the cationically modified poly alpha-1,6-glucan ether compound has a weight average molecular weight of from about 50,000 to about 500,000 daltons.
K. A personal care composition according to Paragraph A-J, wherein the cationically modified poly alpha-1,6-glucan ether compound has a charge density of 0.05 to 3 meq/gm.
L. A personal care composition according to Paragraph A-K, wherein the one or more cationic polymer has a charge density (CD) of from about 0.05 to about 7.0 meq/gm.
M. A personal care composition according to Paragraph A-L, wherein the one or more cationic polymer has a charge density (CD) of from about 0.7 to about 3.0 meq/gm.
N. A personal care composition according to Paragraph A-M, wherein the one or more cationic polymer has a molecular weight (MW) of from about 300,000 to about 2,600,000 g/mol.
O. A personal care composition according to Paragraph A-N, wherein the one or more cationic polymer has a molecular weight (MW) of from about 700,000 to about 2,600,000 g/mol.
P. A personal care composition according to Paragraph A-O, wherein the composition has at least a 10% increase in deposition when compared to a composition with to a control composition with no cationically modified poly alpha-1,6-glucan ether compound.
Q. A personal care composition according to Paragraph A-P, wherein the composition has at least a 20% increase in deposition when compared to a composition with to a control composition with no cationically modified poly alpha-1,6-glucan ether compound.
R. A personal care composition according to Paragraph A-Q, wherein the one or more cationic polymer has a molecular weight (MW) of from about 700,000 to about 1,500,000 g/mol and a charge density (CD) of from about 0.5 to about 1.6 meq/gm.
S. A personal care composition according to Paragraph A-R, wherein the anionic detersive surfactant is select from alkyl sulfates, alkyl ether sulfates, acyl glycinates, acyl sarcosinates, acyl glutamates, acyl alaninates, sulfosuccinates, isethionates, sulfonates, sulfoacetates, glucose carboxylates, alkyl ether carboxylates, acyl taurates, sodium, ammonium or potassium salts of these, and mixtures thereof.
T. The personal care composition according to Paragraph A-S, wherein the co-surfactant is selected from aliphatic secondary and tertiary amines, wherein at least one aliphatic substituent contains 8 to 18 carbon atoms and at least one aliphatic substituent contains an anionic selected from carboxy, sulfonate, sulfate, phosphate, and phosphonate.
U. The personal care composition according to Paragraph A-T, wherein the co-surfactant is selected from cocoamphoacetate, cocoamphodiacetate, lauroamphoacetate, lauroamphodiacetate, and mixtures thereof.
V. The personal care composition according to Paragraph A-U, wherein the co-surfactant is selected from derivatives of aliphatic quaternary ammonium, phosphonium, and sulfonium compounds, wherein at least one aliphatic substituent contains 8 to 18 carbon atoms and at least one aliphatic substituent contains an anionic group selected from carboxy, sulfonate, sulfate, phosphate and phosphonate.
W. The personal care composition according to paragraph A-V, wherein the co-surfactant is selected from cocamidopropyl betaine, lauramidopropyl betaine and mixtures thereof.
X. A personal care composition according to Paragraph A-W, further comprising from about 0.25% to about 15% of the co-surfactants.
Y. A personal care composition according to Paragraph A-X, wherein the one or more cationic polymers are selected from the group consisting of a cationic guar polymer, a cationic non-guar galactomannan polymer, a cationic tapioca polymer, a cationic copolymer of acrylamide monomers and cationic monomers, a synthetic, non-crosslinked, cationic polymer, which may or may not form lyotropic liquid crystals upon combination with the detersive surfactant, a cationic cellulose polymer and mixtures thereof.
Z. A personal care composition according to Paragraph A-Y, wherein the scalp care active selected from the group consisting of hydroxyl pyridone, azoles, climbazole, ketoconazole, salicylic acid, polyvalent metal salts of pyrithione, sulfur, and selenium sulfide.
AA. A personal care composition according to Paragraph A-Z, wherein the hydroxyl pyridone is piroctone olamine.
BB. A personal care composition according to Paragraph A-AA, wherein the polyvalent metal salt of pyrithione is zinc pyrithione.
CC. A personal care composition according to Paragraph A-BB, wherein the composition has at least a 1× increase in deposition when compared to a composition with to a control composition with no cationically modified poly alpha-1,6-glucan ether compound.
DD. A personal care composition according to Paragraph A-CC, wherein the composition has at least a 2× increase in deposition when compared to a composition with to a control composition with no cationically modified poly alpha-1,6-glucan ether compound. EE. A personal care composition according to Paragraph A-DD, wherein there is an increase in coacervation as measured by a decreasing % T and resulting in an increase in scalp care active deposition compared to control composition with no cationically modified poly alpha-1,6-glucan ether compound.
FF. A personal care composition according to Paragraph A-EE, comprising
   a. from about 8 to about 20% of one or more surfactants;
   b. from about 0.01 to about 10% of scalp care active;
   c. from about 0.01 to about 5% of one or more cationic polymer;
   d. from about 0.1% to about 5% of a cationically modified poly alpha-1,6-glucan ether compound; wherein the composition is free of sulfated surfactants and wherein the composition has an increase in deposition of the scalp care active when compared to a control composition with no cationically modified poly alpha-1,6-glucan ether.

It will be appreciated that other modifications of the present disclosure are within the skill of those in the personal care formulation art can be undertaken without departing from the spirit and scope of this invention. All parts, percentages, and ratios herein are by weight unless otherwise specified. Some components may come from suppliers as dilute solutions. The levels given reflect the weight percent of the active material, unless otherwise specified. A level of perfume and/or preservatives may also be included in the following examples.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A personal care composition comprising
a. from about 8 to about 20% of one or more surfactants;
b. from about 0.01 to about 10% of scalp care active;
c. from about 0.01 to about 5% of one or more cationic polymer;
d. from about 0.1% to about 5% of a cationically modified poly alpha-1,6-glucan ether compound; and wherein the composition has an increase in deposition of the scalp care active when compared to a control composition with no cationically modified poly alpha-1,6-glucan ether compound.

2. A personal care composition according to claim 1, wherein the cationically modified poly alpha-1,6-glucan ether compound:
comprises a cationically modified poly alpha-1,6-glucan substituted with at least one positively charged organic group;
comprises a backbone of glucose monomer units wherein at least 65% of the backbone glucose monomer units are linked via alpha-1,6-glycosidic linkages;
has a degree of substitution of from 0.001 to 3; and
is characterized by one or more of the following i-iii:
i. a weight average degree of polymerization of from 5 to 6000,
ii. a weight average molecular weight of from 1000 to 500,000 daltons,
iii. derived from a poly alpha-1,6-glucan having a weight average molecular weight of from 900 to 450,000 daltons determined prior to substitution with the least one positively charged organic group.

3. A personal care composition according to claim 1, wherein from about 5% to about 35% of the backbone glucose monomer units have branches via alpha-1,2 and/or alpha-1,3-glycosidic linkages.

4. A personal care composition according to claim 2, wherein the positively charged organic group comprises a substituted ammonium group.

5. A personal care composition according to claim 4, wherein the substituted ammonium group comprises a quaternary ammonium group comprising at least one C10 to C16 alkyl group.

6. A personal care composition according to claim 4, wherein the substituted ammonium group comprises a quaternary ammonium group comprising a trimethylammonium group.

7. A personal care composition according to claim 2, wherein the positively charged organic group comprises a quaternary ammonium hydroxyalkyl group.

8. A personal care composition according to claim 1, comprising from about from 0.01% to 5% of the cationically modified poly alpha-1,6-glucan ether compound by weight of the personal care composition.

9. A personal care composition according to claim 1, wherein the cationically modified poly alpha-1,6-glucan ether compound has a degree of substitution of from about 0.01 to about 0.3.

10. A personal care composition according to claim 1, wherein the cationically modified poly alpha-1,6-glucan ether compound has a weight average molecular weight of from about 50,000 to about 500,000 daltons.

11. A personal care composition according to claim 1, wherein the cationically modified poly alpha-1,6-glucan ether compound has a charge density of 0.05 to 3 meq/gm.

12. A personal care composition according to claim 1 wherein the one or more cationic polymer has a charge density (CD) of from about 0.05 to about 7.0 meq/gm.

13. A personal care composition according to claim 1 wherein the one or more cationic polymer has a charge density (CD) of from about 0.7 to about 3.0 meq/gm.

14. A personal care composition according to claim 1 wherein the one or more cationic polymer has a molecular weight (MW) of from about 300,000 to about 2,600,000 g/mol.

15. A personal care composition according to claim 1 wherein the one or more cationic polymer has a molecular weight (MW) of from about 700,000 to about 2,600,000 g/mol.

16. A personal care composition according to claim 1 wherein the composition has at least a 10% increase in deposition when compared to a composition with to a control composition with no cationically modified poly alpha-1,6-glucan ether compound.

17. A personal care composition according to claim 1 wherein the composition has at least a 20% increase in deposition when compared to a composition with to a control composition with no cationically modified poly alpha-1,6-glucan ether compound.

18. A personal care composition according to claim 1 wherein the one or more cationic polymer has a molecular weight (MW) of from about 700,000 to about 1,500,000 g/mol and a charge density (CD) of from about 0.5 to about 1.6 meq/gm.

19. A personal care composition according to claim 1, wherein the anionic detersive surfactant is select from alkyl sulfates, alkyl ether sulfates, acyl glycinates, acyl sarcosinates, acyl glutamates, acyl alaninates, sulfosuccinates, isethionates, sulfonates, sulfoacetates, glucose carboxylates, alkyl ether carboxylates, acyl taurates, sodium, ammonium or potassium salts of these, and mixtures thereof.

20. The personal care composition according to claim 1, wherein the co-surfactant is selected from aliphatic secondary and tertiary amines, wherein at least one aliphatic substituent contains 8 to 18 carbon atoms and at least one aliphatic substituent contains an anionic selected from carboxy, sulfonate, sulfate, phosphate, and phosphonate.

21. The personal care composition according to claim 20, wherein the co-surfactant is selected from cocoamphoacetate, cocoamphodiacetate, lauroamphoacetate, lauroamphodiacetate, and mixtures thereof.

22. The personal care composition according to claim 1, wherein the co-surfactant is selected from derivatives of aliphatic quaternary ammonium, phosphonium, and sulfonium compounds, wherein at least one aliphatic substituent contains 8 to 18 carbon atoms and at least one aliphatic substituent contains an anionic group selected from carboxy, sulfonate, sulfate, phosphate and phosphonate.

23. The personal care composition according to claim 22, wherein the co-surfactant is selected from cocamidopropyl betaine, lauramidopropyl betaine and mixtures thereof.

24. A personal care composition according to claim 1 further comprising from about 0.25% to about 15% of the co-surfactants.

25. A personal care composition according to claim 1 wherein the one or more cationic polymers are selected from the group consisting of a cationic guar polymer, a cationic non-guar galactomannan polymer, a cationic tapioca polymer, a cationic copolymer of acrylamide monomers and cationic monomers, a synthetic, non-crosslinked, cationic polymer, which may or may not form lyotropic liquid crystals upon combination with the detersive surfactant, a cationic cellulose polymer and mixtures thereof.

26. A personal care composition according to claim 1 wherein the one or more cationic polymer is selected from the group consisting of guar hydroxypropyltrimonium chloride, salts of hydroxyethyl cellulose reacted with trimethyl ammonium substituted epoxide, a cationic copolymer of acrylamide monomers and cationic monomers, a synthetic, non-crosslinked, cationic polymer, which may or may not form lyotropic liquid crystals upon combination with the detersive surfactant.

27. A personal care composition according to claim 1 wherein the scalp care active selected from the group consisting of hydroxyl pyridone, azoles, climbazole, ketoconazole, salicylic acid, polyvalent metal salts of pyrithione, sulfur, and selenium sulfide.

28. A personal care composition according to claim 27 wherein the hydroxyl pyridone is piroctone olamine.

29. A personal care composition according to claim 27, wherein the polyvalent metal salt of pyrithione is zinc pyrithione.

30. A personal care composition according to claim 1, wherein the composition has at least a 1× increase in deposition when compared to a composition with to a control composition with no cationically modified poly alpha-1,6-glucan ether compound.

31. A personal care composition according to claim 1, wherein the composition has at least a 2× increase in deposition when compared to a composition with to a control composition with no cationically modified poly alpha-1,6-glucan ether compound.

32. A personal care composition according to claim 1, wherein there is an increase in coacervation as measured by a decreasing % T and resulting in an increase in scalp care active deposition compared to control composition with no cationically modified poly alpha-1,6-glucan ether compound.

33. A personal care composition comprising
a. from about 8 to about 20% of one or more surfactants;
b. from about 0.01 to about 10% of scalp care active;
c. from about 0.01 to about 5% of one or more cationic polymer;
d. from about 0.1% to about 5% of a cationically modified poly alpha-1,6-glucan ether compound; wherein the composition is free of sulfated surfactants and wherein the composition has an increase in deposition of the scalp care active when compared to a control composition with no cationically modified poly alpha-1,6-glucan ether.
